(19) United States
(12) Patent Application Publication
(10) Pub. No.: US 2024/0165266 A1
(43) Pub. Date: May 23, 2024

(54) LIPID NANOPARTICLE FORMULATIONS AND METHODS OF USE THEREOF

(71) Applicant: Board of Regents of the University of Nebraska, Lincoln, NE (US)

(72) Inventors: Howard E. Gendelman, Omaha, NE (US); Jonathan Herskovitz, Omaha, NE (US); Mahmudul Hasan, Omaha, NE (US); Bhavesh Kevadiya, Omaha, NE (US); Milankumar Patel, Omaha, NE (US)

(21) Appl. No.: 18/550,828

(22) PCT Filed: Mar. 17, 2022
(86) PCT No.: PCT/US2022/071207

§ 371 Date: Sep. 15, 2023

Related U.S. Application Data
(60) Provisional application No. 63/262,024, filed on Oct. 1, 2021.
(60) Provisional application No. 63/162,161, filed on Mar. 17, 2021.

(51) Int. Cl. A61K 48/00; A61K 9/127; A61K 31/7105; A61K 38/46; A61K 51/12; A61P 31/18; C12N 9/22; C12N 15/11; C12N 15/88

(52) U.S. Cl. CPC A61K 48/0041; A61K 9/1272; A61K 31/7105; A61K 38/465; A61K 51/1244; A61P 31/18; C12N 9/22; C12N 15/11; C12N 15/88; C12N 2310/20

(57) ABSTRACT

Disclosed herein are methods and compositions for lipid nanoparticles encapsulating a nucleic acid encoding for a CRISPR nucleic acid complementary to a HIV-1 gene. Also disclosed are lipid nanoparticle compositions, nucleotides, cells, and methods associated with the compositions.

### CROSS REFERENCE TO RELATED APPLICATIONS

This application is the National Stage entry of International Application No. PCT/US2022/071207, filed on Mar. 17, 2022, which claims priority to and benefit from U.S. Patent Application No. 63/162,161 filed Mar. 17, 2021, and US Patent Application No. 63/262,024 filed Oct. 1, 2021, each of which is incorporated herein in its entirety.

### STATEMENT AS TO FEDERALLY SPONSORED RESEARCH

This invention was made with government support under Grants No. R01 MH115860, R01 MH121402, R01 AG043540, P01 DA028555, R01 NS36126, P01 NS31492, 2R01 NS034239, P01 MH64570, 3P30 MH062261, P30 AI078498, 1R24 OD018546, and R01 AG043540 all awarded by the National Institutes of Health. The government has certain rights in the invention.

### SEQUENCE LISTING

This application contains a Sequence Listing in computer readable form. The computer readable form is incorporated herein by reference. Said ASCII copy, created on Sep. 13, 2023, is named UNM-003WOUS_SL.txt and is 7,959 bytes in size.

### SUMMARY

Provided herein, in some embodiments, are lipid nanoparticle formulations comprising a plurality of lipids and a CRISPR nucleic acid complementary to a HIV-1 gene. Additionally provided herein, in certain embodiments, are compositions comprising lipid nanoparticle formulations comprising a plurality of lipids and a CRISPR nucleic acid complementary to a HIV-1 gene. Further provided herein, in certain embodiments, are pharmaceutical compositions comprising lipid nanoparticle formulations comprising a plurality of lipids and a CRISPR nucleic acid complementary to a HIV-1 gene, and a pharmaceutically acceptable excipient. Further provided herein, in certain embodiments, are methods for the treatment and prevention of an HIV infection in an individual in need thereof, comprising administering to the individual lipid nanoparticle formulations comprising a plurality of lipids and a CRISPR nucleic acid complementary to a HIV-1 gene.

Disclosed herein, in some embodiments, is a lipid nanoparticle, comprising a plurality of lipids and a CRISPR nucleic acid complementary to a HIV-1 gene.

In some embodiments, the lipid nanoparticle comprises cationic lipids, zwitterionic lipids, cholesterol, and PEG-lipid conjugates.

In some embodiments, the lipid nanoparticle comprises DMG-PEG2500, ionizable lipids, DSPC, cholesterol, and a stabilizer.

In some embodiments, the lipid nanoparticle comprises DSPE-PEG₂₀₀₀ and/or DMP-PEG₂₀₀₀, DOPE, cholesterol, DOTAP.

In some embodiments, the lipid nanoparticle comprises DSPE-PEG2000, DOPE, Cholesterol, DMG-PEG, and DOTAP, and wherein the molar percentages are about 5% to about 15%, about 5% to about 15%, about 20 to about 30%, about 1% to about 5%, and about 40 to about 60%, respectively.

In some embodiments, the lipid nanoparticle comprises a crRNA sequence that is complementary to a plurality of nucleic acids of a consensus sequence of an HIV-1 gene selected from the group consisting of: tat, rev, env-gp41, gag-p1, gag-p6, vif, vpr, vpu, and nef.

In some embodiments, the nucleic acid sequence comprises two crRNA sequences, each sequence complementary to a plurality of nucleic acids of a consensus sequence of an HIV-1 gene selected from the group consisting of: tat, rev, env-gp41, gag-p1, gag-p6, vif, vpr, vpu, and nef: wherein the crRNA sequences are not complementary to the same sequences.

In some embodiments, the crRNA sequence is adjacent to a PAM sequence.

In some embodiments, the crRNA sequence is complementary to a plurality of nucleic acids of an overlapping sequence.

In some embodiments, the overlapping sequence is part of a nucleic acid sequence of at least two HIV-1 genes selected from the group consisting of: tat, rev, env-gp41, gag-p1, gag-p6, vif, vpr, vpu, and nef.

In some embodiments, the overlapping sequence is part of a nucleic acid sequence of at least three HIV-1 genes selected from the group consisting of: tat, rev, env-gp41, gag-p1, gag-p6, vif, vpr, vpu, and nef.

In some embodiments, the overlapping exon is part of a nucleic acid sequence selected from the group consisting of tat (exon 1, nucleic acids 5831-6045; exon 2, nucleic acids 8379-8469), rev (exon 1, nucleic acids 5970-6045: or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417).

In some embodiments, the overlapping sequence is nucleic acids 7758-8795 of HIV-1 gene gp41-env, exon 2 (nucleic acids 8379-8469) of HIV-1 gene tat, and exon 2 (nucleic acids 8379-8653) of HIV-1 gene rev.

In some embodiments, the overlapping exon is exon 1 (nucleic acids 5831-6045) of HIV-1 gene tat, and exon 1 (nucleic acids 5970-6045) of HIV-1 gene rev.

In some embodiments, the crRNA has a sequence at least 80%, 85%, 90%, or 95% identical to SEQ ID NO: 1.

In some embodiments, the crRNA has a sequence at least 80%, 85%, 90%, or 95% identical to SEQ ID NO: 2.

In some embodiments, the crRNA has a sequence at least 80%, 85%, 90%, or 95% identical to SEQ ID NO: 3.

In some embodiments, the crRNA has a sequence at least 80%, 85%, 90%, or 95% identical to SEQ ID NO: 4.

In some embodiments, the crRNA has a sequence at least 80%, 85%, 90%, or 95% identical to SEQ ID NO: 5.

In some embodiments, the crRNA has a sequence at least 80%, 85%, 90%, or 95% identical to SEQ ID NO: 6.

In some embodiments, the crRNA has a sequence at least 80%, 85%, 90%, or 95% identical to SEQ ID NO: 7.

In some embodiments, the crRNA has a sequence at least 80%, 85%, 90%, or 95% identical to SEQ ID NO: 8.

In some embodiments, the crRNA has a sequence according to SEQ ID NO: 1.

In some embodiments, the crRNA has a sequence according to SEQ ID NO: 2.

In some embodiments, the crRNA has a sequence according to SEQ ID NO: 3.

In some embodiments, the crRNA has a sequence according to SEQ ID NO: 4.

In some embodiments, the crRNA has a sequence according to SEQ ID NO: 5.

In some embodiments, the crRNA has a sequence according to SEQ ID NO: 6.

In some embodiments, the crRNA has a sequence according to SEQ ID NO: 7.

In some embodiments, the crRNA has a sequence according to SEQ ID NO: 8.

In some embodiments, the nucleic acid encodes for a TatDE crRNA.

In some embodiments, the TatDE crRNAs comprise SEQ ID NO: 2 and SEQ ID NO: 3.

In some embodiments, the nucleic acid sequence further comprises a tracrRNA sequence.

In some embodiments, the nucleic acid sequence further comprises a sequence that encodes a Cas protein.

In some embodiments, the Cas protein is a Cas9, CasPhi (Cas Φ), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas12a (Cpf1), Cas12b, Cas12e, Cas13a, Cas13, Cas13c, or Cas13d.

In some embodiments, the Cas protein is a Cas9) protein.

In some embodiments, the nucleic acid encoding for Cas9 is a vector and the nucleic acid encoding for TatDE crRNAs is a vector.

In some embodiments, the nucleic acid encoding for Cas9 is a mRNA and the nucleic acid encoding for TatDE crRNAs is a mRNA.

In some embodiments, the nucleic acid sequence is a DNA sequence.

In some embodiments, the nucleic acid sequence is a RNA sequence.

Also provided herein, in some embodiments, is a pharmaceutical composition, comprising (a) the lipid nanoparticle disclosed herein, and (b) a pharmaceutically acceptable excipient.

In some embodiments, the pharmaceutical composition comprises (a) a nucleic acid comprising TatD and (b) a nucleic acid comprising TatH (TatD/H).

In some embodiments, the pharmaceutical composition comprises (a) a nucleic acid comprising TatD and (b) a nucleic acid comprising TatE (TatD/E).

In some embodiments, the pharmaceutical composition comprises (a) a nucleic acid comprising TatE and (b) a nucleic acid comprising TatH (TatE/H).

In some embodiments, the pharmaceutical composition comprises (a) a nucleic acid comprising TatD and (b) a nucleic acid comprising TatA₂ (TatA₂/D).

In some embodiments, the pharmaceutical composition comprises (a) a nucleic acid comprising TatD/tracrRNA and (b) a nucleic acid comprising TatH/tracrRNA.

In some embodiments, the pharmaceutical composition comprises (a) a nucleic acid comprising TatD/tracrRNA and (b) a nucleic acid comprising TatE/tracrRNA,

In some embodiments, the pharmaceutical composition comprises (a) a nucleic acid comprising TatE/tracrRNA and (b) a nucleic acid comprising TatH/tracrRNA.

In some embodiments, the pharmaceutical composition comprises (a) a nucleic acid comprising TatD/tracrRNA and (b) a nucleic acid comprising TatA₂/tracrRNA.

Also provided herein, in some embodiments, is a method of disrupting the transcription of an exon of an HIV-1 sequence in an individual in need thereof, comprising administering to the individual the lipid nanoparticle disclosed herein or the pharmaceutical composition disclosed herein.

Also provided herein, in some embodiments, is a method of excising all or a portion of an HIV-1 sequence in an individual in need thereof, comprising administering to the individual the lipid nanoparticle disclosed herein or the pharmaceutical composition disclosed herein.

Also provided herein, in some embodiments, is a method of treating an HIV-1 infection in an individual in need thereof, comprising administering to the individual the lipid nanoparticle disclosed herein or the pharmaceutical composition disclosed herein.

Also provided herein, in some embodiments, is a method of preventing, treating, and/or eradicating a disease in a subject in need thereof, said method comprising administering to said subject a lipid nanoparticle disclosed herein or the pharmaceutical composition disclosed herein.

Also provided herein, in some embodiments, is a method of preventing an HIV-1 infection in an individual in need thereof, comprising prophylactically administering to the individual the lipid nanoparticle disclosed herein or the pharmaceutical composition disclosed herein.

Also provided herein, in some embodiments, is a method of preventing transmission of an HIV-1 virus from a first individual to a second individual, comprising administering to the first individual the lipid nanoparticle disclosed herein or the pharmaceutical composition described herein.

In some embodiments, the first individual is a pregnant woman and the second individual is a child.

Also provided herein, in some embodiments, are lipid nanoparticles, further comprising a diagnostic agent.

In some embodiments, the diagnostic agent is a MRI contrast agent, a fluorescent dye, or a nuclear medicine agent.

In some embodiments, the diagnostic agent is a radiolabeled europium doped cobalt ferrite nanoparticle (177Lu/89ZrCFEu nanoparticle).

In some embodiments, disclosed herein is a method of diagnosing an individual in need of HIV-1 therapy, comprising administering to the individual a lipid nanoparticle disclosed herein which further comprises a diagnostic agent or a pharmaceutical composition comprising a lipid nanoparticle disclosed herein which further comprises a diagnostic agent and a pharmaceutically acceptable excipient.

Other objects and advantages will become apparent to those skilled in the art from a consideration of the ensuing Detailed Description, Examples, and Claims.

### DESCRIPTION OF THE DRAWINGS

FIGS. 1A-C illustrates CRISPR-Cas9 nanoparticle synthesis. FIG. 1A shows an exemplary schematic for preparation of radiolabeled europium doped cobalt ferrite nanoparticles (¹⁷⁷Lu/⁸⁹ZrCFEu). The particles were manufactured by a modified solvothermal technique. Lutetium-177 or Zirconium-89 were made containing Iron (III) acetylacetonate, cobalt (II) acetylacetonate and europium (III) nitrate pentahydrate. The color graphical descriptions are as follow. Red spheres are iron; blue spheres are cobalt and pink spheres are europium. These are dissolved by sonication in benzyl alcohol (as the solvent) in the presence of reducing and stabilizing agents 1,2-hexadecanediol, oleic acid and oleamine. Upon nucleation reaction in a hydrothermal autoclave reactor, the nanoparticles were purified by ethanol and centrifugations. FIG. 1B shows an exemplary schematic for radiolabeled prodrug made in lipid nanoparticles (LNPs). Microfluidic techniques was used to synthesize LNPs containing the cabotegravir prodrug (M2CAB) and rilpivirine (M3RPV) with the bioimaging agent 177Lu/89ZrCFEu. LNP synthesis included cholesterol. PEG-lipids (DSPE-PEG2000, and DMG-PEG), zwitterionic lipid (DOPE), and anionic lipid (12:0 PG). Lipid mixtures, prodrugs, and radiolabeled nanoparticles were passed through microfluidic microchannels under controlled pressures and flow rates to prepare the radiolabeled prodrug lipid nanoparticles (M2CAB/M3RPV@¹⁷⁷Lu/⁸⁹ZrCFEu). The loaded LNPs were purified by dialysis. FIG. 1C shows an exemplary schematic for preparation of radiolabeled CRISPR-Cas9 plasmid and ribonucleoprotein (RNP) LNPs.

FIGS. 2A-J illustrates the synthesis, characterization and antiretroviral activity of CRISPR-Cas9 lipid nanoparticles (LNPs) in primary human monocyte-derived macrophage (MDM). FIG. 2A shows exemplary process for CRISPR-Cas9 TatDE LNPs prepared by thin film hydration by mixing cholesterol. PEG-lipids (DSPE-PEG2000, and DMG-PEG), zwitterionic lipid (DOPE) and cationic lipid (DOTAP) with the CRISPR-Cas9) TatDE plasmid. The prepared mixture was dialyzed prior to virologic testing. FIG. 2B shows exemplary transmission electron microscopy (TEM) images of the CRISPR-Cas9 loaded LNPs. The scale bar is 100 nm. FIG. 2C shows exemplary atomic force microscopy (AFM) topographic images of the loaded LNPs demonstrate average height profiles. FIG. 2D shows an exemplary dynamic light scattering (DLS) graph that illustrates the size distribution of CRISPR-Cas9 loaded LNPs with an average size of 180 nm (PDI=0.2). FIG. 2E shows exemplary ethidium bromide (EtBr)-stained LNPs(i) fluorescing under ultraviolet (UV) excitation compared against unstained LNPs(ii). FIG. 2F shows RT activity over time for MDM treated with the CRISPR-Cas9 LNPs at a concentration of 100-400 ng TatDE particles/cell then challenged with HIV-1 ADA (macrophage tropic viral strain) at a multiplicity of infection (MOI) of 0.01 infectious viral particles/cell. HIV-1 infection was monitored by levels of reverse transcriptase (RT) activity reflective of progeny virions in culture fluids for a time period of 7 days. FIG. 2G shows exemplary image related to polymerase chain reaction (PCR) was performed in cell lysates followed by agarose gel electrophoresis. This confirmed protection was size of the “putative” virus-excised band.

FIG. 2H shows the sized band was confirmed as the appropriate CRISPR-Cas9 excised subgenomic viral DNA by Sanger sequencing by the multiple alignment program for nucleotide sequences (MAFFT). FIG. 2I shows RT activity over time for MDM treated with the CRISPR-Cas9 LNPs at a concentration of 100-400 ng TatDE equivalent then challenged with HIV-1 ADA (macrophage tropic viral strain) at an MOI of 0.02. HIV-1 replication was monitored by RT activity in culture fluids as an indication of progeny virions for up to 7 days. Statistical significance was calculated using one way ANOVA for multiple comparisons (n=3) with Dunnets post hoc test. For 400 ng concentration. P=0.01; for 200 ng concentration P=0.04. PCR was performed in cell lysates using HIV-1 specific primers, followed by agarose gel electrophoresis. The excised band at ˜2.4 kb is highlighted in yellow confirms “putative” protection. FIG. 2J shows exemplary transmission electron microscopy images of the CRISPR-Cas9 loaded LNPs showed spherical morphology including a particle surface corona. The scale bar is 100 nm.

FIGS. 3A-C illustrates HIV CRISPR-Cas9 Mosaic gRNA Design. FIG. 3A shows nucleotide heterogeneity of 4004 annotated HIV-1 strains depicted in a heat-map form demonstrating entropic (blue) or conserved (red) loci in three reading frames. Prior reported gRNAs against LTR and gag regions were used as reference controls FIG. 3B shows designed gRNAs targeting mosaic HIV-1tat sequences, and FIG. 3C shows for antisense or sense sequences are shown by down or upward facing arrows, respectively.

FIGS. 4A-D illustrates TatDE gRNAs Facilitate Multistrain HIV-1 Excision. FIG. 4A shows a gRNA library was screened against a panel of HIV-1 molecular clones by co-transfection into HEK293FT cells. Progeny virion production was measured by reverse transcriptase (RT) activity in culture fluids. FIG. 4B shows a Pearson correlation between gRNA target conservation among 4004 proviral DNA sequences and RT knockdown were assessed. FIG. 4C shows PCR tests were completed on DNA extracted from amplified untreated or CRISPR-TatDE plasmid-treated cells. The white arrow indicates the expected molecular size of the TatDE excision band. FIG. 4D shows PCR reaction contents were Sanger sequenced and evaluated in Inference of CRISPR Edits v2.0 (ICE. Synthego 2020) to visualize nucleotide editing in the PAM/protospacer regions. Data in (a-b, d) depict mean±standard error of the mean (SEM) from four independent experiments. Each of the experiments were performed in triplicate.

FIGS. 5A-E illustrates lentiviral TatDE CRISPR Inactivates Latent HIV-1. ACH2 T cells bearing a single copy of HIV-1 proviral DNA were transduced with lentivirus bearing a spCas9-gRNA transgene at multiplicities of infection (MOI) of 10, 1, or 0.1. After 72 hours, cells were stimulated with tumor necrosis factor alpha (TNFα, 15 ng/ml) for 72 hours. FIG. 5A shows spCas9 expression was measured by RT-qPCR. FIGS. 5B-D show RT activity recorded from culture supernatant fluids. FIG. 5E shows nested PCR for assayed proviral DNA excision wherein unedited amplicons are 2986 bp and CRISPR-edited amplicons are approximately 525 bp. These differences are dependent on insertion-deletion mutagenesis. The arrow indicates the expected molecular size in the presence of TatDE excision gRNAs. Significance was determined by a two-way ANOVA.

FIGS. 6A-E illustrates Exonic Disruption and HIV-1 Replicative Fitness. FIGS. 6A-B show insertion-deletion profiles among the generated gRNAs obtained through a co-transfection screen were assessed by the Synthego ICE v2.0 algorithm. The highest frequency insertions or deletions were selected for subsequent non-frameshift site-directed mutagenesis of the HIV-1_{NL4-3-Δnef-eGFP} encoding plasmid. FIGS. 6A-6B discloses SEQ ID NOS 17-30, respectively, in order of appearance. FIG. 6C shows exemplary transmission electron micrographs of single- or dual-tat mutants are illustrated. Spherical diameter measurements were taken (inset). FIGS. 6D-E show CEMss CD4+ T cell lines were challenged with HIV-1_{NL4-3-Δtat-Δnef-cGFP} at an MOI of MOI 0.1 and assayed at defined time points for RT activity (FIG. 6D). Flow cytometry assay results for % GFP-positive cells are shown in FIG. 6E.

FIGS. 7A-D illustrates CRISPR LNPs cell trafficking. Rhodamine DHPE phospholipid tracked the locale of CRISPR LNPs in human MDMs. Confocal microscopy was employed 12 h after particle injection in the MDM cultures. Alexa-Fluor 488 (green) secondary antibody detected Rab 5. Rab7, or Lamp1 subcellular compartments. Phalloidin-iFluor 647 marked cell boundaries. The MDM nucleus was stained with DAPI (blue). Rhodamine DHPE phospholipid containing CRISPR-LNPs (red) colocalized with Rab5 (FIG. 7A) and Rab7 (green) (FIG. 7B). FIG. 7C shows no-colocalization was found between Lamp1 (green) and the nanoparticles (red). (FIG. 7D) TM-Rhodamine labeled px333DE was used for CRISPR LNPs to examine nuclear localization of the CRISPR payload present in the nucleus 12h after treatment. Z-stack affirmed that the CRISPR reached the nucleus.

FIGS. 8A-G Illustrates HIV-1 RNP Delivery for Virus Editing. FIG. 8A shows TatD/TatE RNPs were assembled then co-transfected with two infectious HIV-1 molecular clones by TransIT-X2 transfection into HEK 293FT cells to determine Cas9 efficacy. FIG. 8B shows measurements in supernatants from transfected cells show that the HIV-1 RNP treatment reduces virion production to or around control levels. For NL4-3. P=0.02, for pCH040.c/2625 P=0.01 by unpaired t test compared to untreated infected control. FIG. 8C shows DNA PCR tests from the HIV-1 proviral clones show that all HIV-1 DNA was cleaved. FIG. 8D shows cell vitality MTT assay performed on the electroporated cells showed no significant change in cell viability. One-way ANOVA (n=3) with Dunnet's post-hoc test showed non-significant differences between different groups: P=0.54 and F=0.77. After 72 hours following electroporation. ACH2 cells were stimulated with TNF-α (15 ng/ml). FIG. 8E shows the efficacy of TatD/TatE RNPs were tested for viral excision in latent HIV-1 infected ACH2 cells. These cells carry a single copy of proviral DNA. RNPs were delivered to the ACH2 infected cells by electroporation. FIG. 8F shows tests for RT activity in culture fluids showed that the RNP treated ACH2s upon stimulation did not produce progeny virus. Untreated control cells showed significantly higher fold stimulation; P=0.01 by unpaired t-test. Then, viral excision was analyzed by DNA PCR. FIG. 8G shows PCR tests showed intact viral genome (3025 bp) in untreated controls, whereas full length HIV-1 proviral DNA was not detected in the treated groups. An expected 525 bp excised amplicon was readily seen in both stimulated and unstimulated RNP treated cells. Data points in FIG. 8B. FIG. 8D and FIG. 8F depict mean±SEM from biological triplicates.

FIGS. 9A-G illustrates mRNA Loaded TatDE LNPs. FIG. 9A shows an exemplary schematic representation of the LNP components and the manufacturing process using non turbulent microfluidic mixing. FIG. 9B shows LNPs loaded with CleanCap Firefly luciferase (Fluc) mRNA and Dasher GFP mRNA showed high encapsulation efficiency of 94.4% and 83.8% quantified by the Ribogreen RNA assay kit. FIGS. 9C-D show Fluc and GFP LNPs had a very narrow size distribution with PDI of 0.083 and 0.098 respectively. They had an average diameter of 73.08 nm and 76.68 nm. U1 and JLat cells were treated with Fluc LNP at 2 μg mRNA equivalent/million cells dose. Forty eight hours following treatment cells were lysed and a luciferase assay was performed. FIG. 9E shows both cell lines show robust luminescence upon addition of Luciferase substrate to cell lysate confirming expression of luciferase delivered by LNP. FIG. 9F shows GFP LNP treatment to the cells shows high GFP expression in both U1 and JLat cell lines affirmed by shift of population from GFP dim to GFP positive cells. FIG. 9G shows eighty six and a half percent of the gated live singlet U1 cells were positive for GFP expression on an average and ≥98% of the gated live singlet JLat 8.4 cells showed GFP expression. Control cells showed less than 0.1% GFP expression. All the treatment experiments (GFP or FLuc) were performed in triplicates.

FIGS. 10A-I illustrates that LNP Cargos HIV-1 TatDE gRNA and Cas9 mRNA attenuate viral replication. CleanCap Cas9 mRNA, TatD and TatE sgRNA was combined and formulated using optimized lipid mix aided by microfluidic mixing to formulate TatDE plasmid LNP (pLNP). They were characterized and tested for anti-viral efficacy. FIG. 10A shows an exemplary schematic representation of antiviral efficacy and toxicity testing. FIG. 10B shows that TatDE pLNP show a narrower size distribution with a PDI of 0.045 and average diameter of 76.11 nm. FIG. 10D shows they also had a high encapsulation efficiency of 92.9%, TatDE pLNP was added to cells at 2 ug Cas9 mRNA equivalent per million of U1 or JLat 8.4 cells. FIG. 10C shows 72 hours post treatment cells were tested for LNP mediated toxicity using MTT vitality assay, TatDE pLNP was non-toxic for the U1 cells (˜100% vitality in treated group) and JLat cells (≥85% vitality in treated group) compared to untreated controls. U1 and JLat 8.4 cells contain one or more integrated copy of viral genome in each cell. Cell genomic DNA were isolated and nested PCR was performed with HIV specific primers. Agarose gel electrophoresis of PCR product showed in both U1 cells (FIG. 10E) and JLat cells (FIG. 10F) full length HIV was present in untreated cells but not treated cells. Treated cells rather had an expected 525 bp excised fragment. PCR was ran from three independent samples. Subsequently treated cells were stimulated with 20 ng/ml TNF-α (JLat 8.4 cells) or 50 nM pMA (U1 cells). 72 hours post stimulation, cells were harvested, and RNA was extracted. FIGS. 10G-H show highly sensitive digital droplet PCR showed induction of HIV RNA production in untreated cells whereas in case of treated groups even after stimulation HIV RNA production was near baseline. FIG. 10I shows that stimulation causes approximately 100 (JLat) to 300 (U1) fold increase in RNA production in untreated cells but almost baseline level stimulation was seen in case of treated groups.

FIG. 11 shows an exemplary experimental scheme for HIV-1 excision by TatDE rLNP delivery.

FIG. 12 shows exemplary images of human HLA-DR expression in spleen confirms human cell reconstitution in all animals (top plates). Replicate sections were stained for HIV-Ip24 and show large numbers of infected cells (bottom panels) in infected animals but not in infected animals treated with ART or ART and CRISPR. Scale (10 μm).

FIG. 13 illustrates excision of HIV-1 DNA by CRISPR-Cas9 in HIV-1 infected humanized mice. The illustration of proviral HIV-INL4-3 DNA highlighting the positions of gRNA LTR1 and gRNA target sites, and the CRISPR-Cas9 induced break points. Total DNA from spleen with primers sets derived the HIV-1 gag gene. Predicted amplicons of 2859 bp and 419 bp, which result from the full length (upper arrows) and excised (lower arrows) HIV-1 DNA fragments are illustrated. The later fragment represents excision of components of the proviral genome. HIV-1 infected animals 941, 956, 958, and 965 were CRISPR-Cas9 treated with or without ART showed absent or reduced full length HIV-1 amplicon (upper arrow) and a present excised (419) (lower arrow) subgenomic viral DNA fragment. Infected animals without evidence of viral excision seen with full length viral amplicons (animals 927, 942, 954, 957, 959 and 960). The spacing of the animal blots were made as the samples were blinded to the participating investigator.

### DETAILED DESCRIPTION

Disclosed herein, in certain embodiments, are lipid nanoparticle formulations comprising a plurality of lipids and a CRISPR nucleic acid complementary to a HIV-1 gene. Further disclosed herein, in certain embodiments, are compositions comprising lipid nanoparticle formulations comprising a plurality of lipids and a CRISPR nucleic acid complementary to a HIV-1 gene. Further disclosed herein, in certain embodiments, are pharmaceutical compositions comprising lipid nanoparticle formulations comprising a plurality of lipids and a CRISPR nucleic acid complementary to a HIV-1 gene, and a pharmaceutically acceptable excipient. Additionally, disclosed herein, in certain embodiments, are methods for the treatment and prevention of an HIV infection in an individual in need thereof, comprising administering to the individual lipid nanoparticle formulations comprising a plurality of lipids and a CRISPR nucleic acid complementary to a HIV-1 gene.

### Definitions

Unless defined otherwise, all technical and scientific terms used herein have the same meaning as commonly understood by one of skill in the chemical and biological arts.

Additionally, as used in this specification and the appended claims, the singular forms “a”. “an” and “the” include plural referents unless the content clearly dictates otherwise. Thus, for example, reference to “an antibody” includes a plurality of antibodies and reference to “an antibody” in some embodiments includes multiple antibodies, and so forth. Furthermore, unless specifically stated otherwise, the term “about” refers to a range of values plus or minus 20% for percentages (i.e., 20% below that number to 20% above that number), typically 10% for percentages (i.e., 10% below that number to 10% above that number) and plus or minus 1.0 unit for unit values, for example, about 1.0 refers to a range of values from 0.9 to 1.1. “About” a range refers to 10% below the lower limit of the range, spanning to 10% above the upper limit of the range.

As used herein. “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide. 2-hydroxy-ethanesulfonate, lactobionate, lactate, laurate, lauryl sulfate, malate, maleate, malonate, methanesulfonate. 2-naphthalenesulfonate, nicotinate, nitrate, oleate, oxalate, palmitate, pamoate, pectinate, persulfate. 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “crRNA” means a non-coding short RNA sequence which bind to a complementary target DNA sequence. The crRNA sequence binds to a Cas enzyme (e.g., Cas9) and the crRNA sequence guides the complex via pairing to a specific target DNA sequence.

As used herein, the term “tracrRNA” or trans-activating CRISPR RNA means an RNA sequence that base pairs with the crRNA (to form a functional guide RNA (gRNA)). The tracrRNA sequence binds to a Cas enzyme (e.g., Cas9), while the crRNA sequence of the gRNA directs the complex to a target sequence.

As used herein, the term “gRNA” means the crRNA and a tracrRNA bound together. The gRNA binds to a Cas enzyme (e.g., Cas9) and guides the Cas enzyme to the target sequence.

As used herein, the term “sgRNA” means a single RNA construct comprising a crRNA sequence and a tracrRNA sequence.

As used herein, the term “mosaic crRNAs” mean crRNAs that are constructed from a multiple sequence alignment of separate viral strains, for example separate HIV-1 strains (92UG_029, KER2008, 99KE_KNH1135 etc) or HIV-2 strains.

As used herein, the term “overlapping sequence” or “overlapping exon” means exons or genes that are transcribed in different reading frame from the same part of the DNA sequence.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

### Lipid Nanoparticles

Disclosed herein, in certain embodiments, are lipid nanoparticles comprising a plurality of lipids and a CRISPR nucleic acid complementary to a HIV-1 gene.

In some embodiments, the compositions of the present invention comprises lipid-based nanoparticles. In some embodiments, the lipid nanoparticles of the present invention comprise one or more lipids. In some embodiments, the lipid nanoparticles further comprises one or more lipid layers. In some embodiments, the lipid nanoparticles comprises a therapeutic agent coated with one or more lipid agents. In some embodiments, the lipid nanoparticles comprises a therapeutic agent coated with one or more lipid agents, which is further coated by one or more additional lipid agents. In some embodiments, the lipid nanoparticles are formed using a variety of lipids including, but not limited to, cationic lipids, anionic lipids, zwitterionic (neutral) lipids, cholesterols, non-polar lipids and lipids modified by other agents or compounds or linked to other agents or compounds including, but not limited, to polymers, or a combination thereof.

Any lipid or combination of lipids that are known in the art can be used to produce a LNP. Examples of lipids used to produce LNPs include, but are not limited to, DOTMA (1,2-di-O-octadecenyl-3-trimethylammonium propane), DOSPA (N-(1-(2,3-dioleyloxy)propyl)-N-2-(sperminecarboxamido)ethyl)-N,N-dimethylammonium trifluoracetate), DOTAP (1,2-dioleoyl-3-trimethylammonium propane), DMRIE (N-(1,2-dimyristyloxyprop-3-yl)-N,N-dimethyl-N-hydroxyethyl ammonium), DC-cholesterol (3β-(N-(N′,N′-dimethylaminoethane)-carbamoyl)cholesterol), DOTAP-cholesterol (1,2-dioleoyl-3-trimethylammonium propane; (3S,8S,9S, 10R, 13R, 14S, 17R)-10,13-dimethyl-17-[(2R)-6-methylheptan-2-yl]-2,3,4,7,8,9,11,12,14,15,16,17-dodecahydro-1H-cyclopenta[a]phenanthren-3-ol), GAP-DMORIE-DPyPE (Vaxfectin: (+)-N-(3-aminopropyl)-N,N-dimethyl-2,3-bis(cis-9)-tetradeceneyloxy)-1-propanaminium: 1,2-diphytanoyl-sn-glycero-3-phosphoethanolamine), and GL67A (GL67-DOPE-DMPE-polyethylene glycol (PEG) (cholest-5-en-3-ol (3β)-,3-[(3-aminopropyl)[4-[(3-aminopropyl)amino]butyl]carbamate: 1,2-dileoyl-sn-3-phosphoethanolamine; dimyristoylphosphoethanolamine; PEG) and pharmaceutically acceptable salts thereof.

Cationic lipids include, but are not limited to, 1,2-di-O-octadecenyl-3-trimethylammonium propane (DOTMA), N,N-dioleyl-N,N-dimethylammonium chloride (DODAC), didodecyldimethylammonium bromide (DDAB), N, N-dimethyl2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP). 1,2-Dilinoleyoxy-3-(dimethylamino)acetoxypropane (DLinDAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl). 1,2-Dilinoleoyl-3-trimethylaminopropane chloride salt (DLin-TAP.Cl), 1,2-Dilinoleyloxy-3-(N-methylpiperazino)propane (DLin-MPZ), 3-(N,N-Dilinoleylamino)-1,2-propanediol (DLinAP), 3-(N,N-Diolcylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), (3aR,5s,6aS)-N,N-dimethyl-2,2-di((9Z,12Z)-octadeca-9,12-dienyl)tetrahydro-3aH-cyclopenta[d][1,3]dioxol-5-amine (ALNY-100), DODAP (1,2-dioleoyl-3-dimethylammonium propane), GL67 (cholest-5-en-3-ol (3β)-, 3-[(3-aminopropyl)[4-[(3-aminopropyl)amino]butyl]carbamate), ethyl PC. DOSPA (N-(1-(2,3-dioleyloxy)propyl)-N-2-(sperminecarboxamido)ethyl)-N,N-dimethylammonium trifluoracetate), DOGS (dioctadecylamidoglycyl carboxyspermine), DORIE (N-(2-hydroxyethyl)-N,N-dimethyl-2,3-bis(((Z)-octadec-9-en-1-yl)oxy)propan-1-aminium), DMRIE (N-(1,2-dimyristyloxyprop-3-yl)-N,N-dimethyl-N-hydroxyethyl ammonium), GAP-DLRIE ((+/−)-N-(3-aminopropyl)-N,N-dimethyl-2,3-bis (dodecyloxy)-1-propanaminium), diC14-amidine, 3ß-[N-(N′,N′-dimethylaminoethane)-carbamoyl]cholesterol (DC-Chol), dimethyldioctadecylammonium (DDA), 1,2-dioleoyl-3-trimethylammonium propane (DOTAP), 1,2-dimyristoyl-3-trimethylammonium-propane (DMTAP), 1,2-stearoyl-3-trimethylammonium-propane (DSTAP) and N-(4-carboxy benzyl)-N,N-dimethyl-2,3-bis(oleoyloxy)propan-1-aminium (DOBAQ), egg phosphatidylcholine, and cholesterol-polyethylene glycol. 98N12-5 (isomer of triethylenetetramine-laurylaminopropionate with a free internal amine, cholesterol, and mPEG2000-C14 glyceride), C12-200 (CAS #: 1220890-25-4; 1,1-((2-(4-(2-((2-(bis(2-hydroxydodecyl)amino)ethyl)(2-hydroxydodecyl) amino)ethyl)piperazin-1-yl)ethyl)azanediyl)bis(dodecan-2-ol)). DLin-KC2-DMA (KC2) (CAS #: 1190197-97-7; 2,2-dilinoleyl-4-(2-dimethylaminoethyl)-[1,3]-dioxolane), DLin-MC3-DMA (MC3) (CAS #: 1224606-06-7; dilinoleylmethyl-4-dimethylaminobutyrate). XTC (2,2-dilinoleyl-4-dimethylaminoethyl-[1,3]-dioxolane), MD1 (CKK-E12; 3,6-bis({4-[bis(2-hydroxy dodecyl)amino]butyl}) piperazine-2,5-dione), 7C1 (C15 epoxide-terminated lipid, e.g., see Dahlman J E, et al. Nat. Nanotechnol. 2014:9(8):648-655), and pharmaceutically acceptable salts thereof.

Examples of zwitterionic (neutral) lipids include, but are not limited to, DSPC (distearoylphosphatidylcholine), dioleoylphosphatidylcholine (DOPC), dioleoylphosphatidylglycerol (DOPG), dioleoyl-phosphatidylethanolamine 4-(N-maleimidomethyl)-cyclohexane-1-carboxylate (DOPE-mal), dipalmitoylphosphatidylglycerol (DPPG), palmitoyloleoylphosphatidylethanolamine (POPE), dipalmitoyl phosphatidyl ethanolamine (DPPE), dimyristoylphosphoethanolamine (DMPE), 16-O-monomethyl PE, 16-O-dimethyl PE, 18-1-trans PE, 1-stearoyl-2-oleoyl-phosphatidyethanolamine (SOPE), DPSC (distearoylphosphatidylcholine), DPPC (dipalmitoylphosphatidylcholine), POPC (palmitoyloleoylphosphatidylcholine), DOPE (1,2-dileoyl-sn-3-phosphoethanolamine), DSPE (1,2-distearoyl-sn-glycero-3-phosphoethanolamine), DMG (dimyristoyl glycerol), phosphatidylserines, phosphatidylethanolamines, phosphatidylcholines, sphingomyelins, sphingophospholipids, betaine lipids (e.g. lauramidopropyl betaine), and SM (sphingomyelin), and combinations thereof.

Anionic lipids may include but are not limited to phosphatidylglycerols (PG), phosphatidic acid and phosphatidylinositol phosphates. Non polar lipids may include but are not limited to glycerides (mono, di, and triglycerides) and other non-charged lipids.

In some embodiments the lipids are modified or conjugated to other molecules. In some embodiments, the lipid is conjugated to a polymer. In some embodiments, the polymer is polyethyelene glycol (PEG). In some embodiments, the PEG has a molecular weight from about 200 g/mol to 10,000 g/mol. In some embodiments, the PEG has a molecular weight from about 200 g/mol to 1.000 g/mol. In some embodiments, the PEG has a molecular weight from about 200 g/mol to 800 g/mol. In some embodiments, the PEG is any molecular weight form of PEG including but not limited to PEG₂₀₀, PEG₃₀₀, PEG₄₀₀, PEG₆₀₀, PEG₁₀₀₀, PEG₂₀₀₀, PEG₃₀₀₀, PEG₆₀₀₀, and PEG₈₀₀₀. Example of PEG-lipid conjugates includes but are not limited to PEG-DMG, PEG-DSPE, PEG-DMP, PEG-CerC14, and PEG-CerC20. In some embodiments, the PEG-lipid conjugates are DMP-PEG₂₀₀₀, DMG-PEG₂₀₀₀ and/or DSPE-PEG₂₀₀₀ or combinations thereof.

In some embodiments, the lipid nanoparticle of the present invention comprises at least one type of cationic lipid. In some embodiments, the lipid nanoparticle of the present invention further comprises at least one type of a zwitterionic lipid. In some embodiments, the lipid nanoparticle of the present invention further comprises at least one PEG-lipid conjugate. In some embodiments, the lipid nanoparticle of the present invention further comprises a cholesterol. In some embodiments, the lipid nanoparticle of the present invention further comprises a therapeutic agent. In some embodiments, the lipid nanoparticle of the present invention further comprises at a diagnostic agent.

In some embodiments, the lipid nanoparticle of the present invention comprises at least one type of a zwitterionic lipid. In some embodiments, the lipid nanoparticle of the present invention further comprises at least one type of a cationic lipid. In some embodiments, the lipid nanoparticle of the present invention further comprises at least one PEG-lipid conjugate. In some embodiments, the lipid nanoparticle of the present invention further comprises a cholesterol. In some embodiments, the lipid nanoparticle of the present invention further comprises a therapeutic agent. In some embodiments, the lipid nanoparticle of the present invention further comprises at a diagnostic agent.

In some embodiments, the lipid nanoparticle of the present invention comprises at least one type of cationic lipid, at least one type of a zwitterionic lipid, and a therapeutic agent. In some embodiments, the lipid nanoparticle of the present invention comprises at least one type of cationic lipid, at least one type of a zwitterionic lipid, at least one PEG-lipid conjugate, a cholesterol, and a therapeutic agent and/or a diagnostic agent.

In some embodiments, the lipid nanoparticle of the present invention comprises DMG-PEG₂₀₀₀ and/or DSPE-PEG₂₀₀₀, DOPE, and DOTAP. In some embodiments, the lipid nanoparticle of the present invention comprises DMG-PEG₂₀₀₀ and/or DSPE-PEG₂₀₀₀, DOPE, DOTAP, and a cholesterol. In some embodiments, the lipid nanoparticle of the present invention comprises DMG-PEG₂₀₀₀ and/or DSPE-PEG₂₀₀₀, DOPE, DOTAP, a cholesterol, and a therapeutic agent. In some embodiments, the lipid nanoparticle of the present invention comprises DMG-PEG₂₀₀₀ and/or DSPE-PEG₂₀₀₀, DOPE, DOTAP, cholesterol, and a therapeutic agent and/or a diagnostic agent.

In some embodiments, the therapeutic agent is an antiviral compound. Exemplary therapeutic agents include, but are not limited to, compounds disclosed in WO/2017/223280. WO/2020/086555, WO/2016/057866, WO/2019/140365, WO/2019/199756, and WO/2020/112931.

In some embodiments, the lipid nanoparticle comprises a cationic lipid in the molar percent of about 30 to about 60%. In some embodiments, the lipid nanoparticle comprises a cationic lipid in the molar percent of about 40 to about 60%. In some embodiments, the lipid nanoparticle comprises a cationic lipid in the molar percent of about 45 to about 55%. In some embodiments, the lipid nanoparticle comprises a cationic lipid in the molar percent of about 40%. In some embodiments, the lipid nanoparticle comprises a cationic lipid in the molar percent of about 45%. In some embodiments, the lipid nanoparticle comprises a cationic lipid in the molar percent of about 50%. In some embodiments, the lipid nanoparticle comprises a cationic lipid in the molar percent of about 55%. In some embodiments, the lipid nanoparticle comprises a cationic lipid in the molar percent of about 60%.

In some embodiments, the lipid nanoparticle comprises a zwitterionic lipid in the molar percent of about 5% to about 35%. In some embodiments, the lipid nanoparticle comprises a zwitterionic lipid in the molar percent of about 5% to about 25%. In some embodiments, the lipid nanoparticle comprises a zwitterionic lipid in the molar percent of about 5% to about 20%. In some embodiments, the lipid nanoparticle comprises a zwitterionic lipid in the molar percent of about 5% to about 15%. In some embodiments, the lipid nanoparticle comprises a zwitterionic lipid in the molar percent of about 10% to about 25%. In some embodiments, the lipid nanoparticle comprises a zwitterionic lipid in the molar percent of about 10% to about 20%. In some embodiments, the lipid nanoparticle comprises a zwitterionic lipid in the molar percent of about 5%. In some embodiments, the lipid nanoparticle comprises a zwitterionic lipid in the molar percent of about 10%. In some embodiments, the lipid nanoparticle comprises a zwitterionic lipid in the molar percent of about 15%. In some embodiments, the lipid nanoparticle comprises a zwitterionic lipid in the molar percent of about 20%. In some embodiments, the lipid nanoparticle comprises a zwitterionic lipid in the molar percent of about 25%. In some embodiments, the lipid nanoparticle comprises a zwitterionic lipid in the molar percent of about 30%.

In some embodiments, the lipid nanoparticle comprises at least one lipid modified with a polymer such as PEG in the molar percent of about 1% to about 30%. In some embodiments, the lipid nanoparticle comprises at least one lipid modified with a polymer such as PEG in the molar percent of about 1% to about 20%. In some embodiments, the lipid nanoparticle comprises at least one lipid modified with a polymer such as PEG in the molar percent of about 5% to about 30%. In some embodiments, the lipid nanoparticle comprises at least one lipid modified with a polymer such as PEG in the molar percent of about 5% to about 20%. In some embodiments, the lipid nanoparticle comprises at least one lipid modified with a polymer such as PEG in the molar percent of about 10% to about 30%. In some embodiments, the lipid nanoparticle comprises at least one lipid modified with a polymer such as PEG in the molar percent of about 10% to about 20%. In some embodiments, the lipid nanoparticle comprises at least one lipid modified with a polymer such as PEG in the molar percent of about 5%. In some embodiments, the lipid nanoparticle comprises at least one lipid modified with a polymer such as PEG in the molar percent of about 10%. In some embodiments, the lipid nanoparticle comprises at least one lipid modified with a polymer such as PEG in the molar percent of about 15%. In some embodiments, the lipid nanoparticle comprises at least one lipid modified with a polymer such as PEG in the molar percent of about 20%. In some embodiments, the lipid nanoparticle comprises at least one lipid modified with a polymer such as PEG in the molar percent of about 25%. In some embodiments, the lipid nanoparticle comprises at least one lipid modified with a polymer such as PEG in the molar percent of about 30%. In some embodiments, the lipid nanoparticle comprises at least two lipids modified with a polymer such as PEG. In some embodiments, the lipid nanoparticle comprises one lipid modified with a polymer such as PEG in the molar percent of about 1% to about 20% and a second lipid modified with a polymer such as PEG in the molar percent of about 0.01% to about 10%. In some embodiments, the lipid nanoparticle comprises one lipid modified with a polymer such as PEG in the molar percent of about 5% to about 20% and a second lipid modified with a polymer such as PEG in the molar percent of about 0.01% to about 5%. In some embodiments, the lipid nanoparticle comprises one lipid modified with a polymer such as PEG in the molar percent of about 5% to about 15% and a second lipid modified with a polymer such as PEG in the molar percent of about 0.5% to about 5%. In some embodiments, the lipid nanoparticle comprises one lipid modified with a polymer such as PEG in the molar percent of about 5% to about 15% and a second lipid modified with a polymer such as PEG in the molar percent of about 1% to about 5%.

In some embodiments, the lipid nanoparticle comprises cholesterol in the molar percent of about 10% to about 40%. In some embodiments, the lipid nanoparticle comprises cholesterol in the molar percent of about 15% to about 40%. In some embodiments, the lipid nanoparticle comprises cholesterol in the molar percent of about 20% to about 40%. In some embodiments, the lipid nanoparticle comprises cholesterol in the molar percent of about 20% to about 30%. In some embodiments, the lipid nanoparticle comprises cholesterol in the molar percent of about 10% to about 30%. In some embodiments, the lipid nanoparticle comprises cholesterol in the molar percent of about 15% to about 30%. In some embodiments, the lipid nanoparticle comprises cholesterol in the molar percent of about 20% to about 40%. In some embodiments, the lipid nanoparticle comprises cholesterol in the molar percent of about 10%. In some embodiments, the lipid nanoparticle comprises cholesterol in the molar percent of about 15%. In some embodiments, the lipid nanoparticle comprises cholesterol in the molar percent of about 20%. In some embodiments, the lipid nanoparticle comprises cholesterol in the molar percent of about 25%. In some embodiments, the lipid nanoparticle comprises cholesterol in the molar percent of about 30%. In some embodiments, the lipid nanoparticle comprises cholesterol in the molar percent of about 35%. In some embodiments, the lipid nanoparticle comprises cholesterol in the molar percent of about 40%.

In some embodiments, the lipid nanoparticle comprises:
a) a cationic lipid in the molar percent of about 40% to about 60%;
b) a zwitterionic lipid in the molar percent of about 1% to about 20%;
c) a lipid modified with a polymer (such as PEG) in the molar percent of about 1% to about 25%; and
e) cholesterol in the molar percent of about 10% to about 40%, wherein the total molar percentage does not exceed 100%.

In some embodiments, the lipid nanoparticle comprises:
a) a cationic lipid in the molar percent of about 40% to about 60%;
b) a zwitterionic lipid in the molar percent of about 1% to about 20%;
c) a first lipid modified with a polymer (such as PEG) in the molar percent of about 1% to about 20%;
d) a second lipid modified with a polymer (such as PEG) in the molar percent of about 0.01% to about 10%; and
e) cholesterol in the molar percent of about 10% to about 40%, wherein the total molar percentage does not exceed 100%.

In some embodiments, the lipid nanoparticle comprises:
a) a cationic lipid in the molar percent of about 40% to about 60%;
b) a zwitterionic lipid in the molar percent of about 5% to about 15%;
c) a lipid modified with a polymer (such as PEG) in the molar percent of about 5% to about 20%; and
e) cholesterol in the molar percent of about 20% to about 30%, wherein the total molar percentage does not exceed 100%.

In some embodiments, the lipid nanoparticle comprises:
a) a cationic lipid in the molar percent of about 40% to about 60%;
b) a zwitterionic lipid in the molar percent of about 5% to about 15%;
c) a first lipid modified with a polymer (such as PEG) in the molar percent of about 5% to about 15%;
d) a second lipid modified with a polymer (such as PEG) in the molar percent of about 1% to about 5%; and
e) cholesterol in the molar percent of about 20% to about 30%, wherein the total molar percentage does not exceed 100%.

In some embodiments, the lipid nanoparticle comprises:
a) DOTAP in the molar percent of about 40% to about 60%;
b) DOPE in the molar percent of about 5% to about 15%;
c) DSPE-PEG2000 in the molar percent of about 5% to about 15%;
d) a DMG-PEGin the molar percent of about 1% to about 5%; and
e) cholesterol in the molar percent of about 20% to about 30%, wherein the total molar percentage does not exceed 100%.

In some embodiments, the lipid nanoparticle comprises:
a) DOTAP in the molar percent of about 45% to about 55%;
b) DOPE in the molar percent of about 5% to about 15%;
c) DSPE-PEG2000 in the molar percent of about 5% to about 15%;
d) a DMG-PEGin the molar percent of about 1% to about 5%; and
e) cholesterol in the molar percent of about 20% to about 30%, wherein the total molar percentage does not exceed 100%.

In some embodiments, the lipid nanoparticle comprises:
a) DOTAP in the molar percent of about 51%;
b) DOPE in the molar percent of about 11%;
c) DSPE-PEG2000 in the molar percent of about 11%;
d) a DMG-PEGin the molar percent of about 3%; and
e) cholesterol in the molar percent of about 24%, wherein the total molar percentage does not exceed 100%.

In some embodiments, the lipid nanoparticle comprises DSPE-PEG2000, DOPE, Cholesterol, DMG-PEG, and DOTAP with molar percent of about 11%, about 11%, about 24%, about 3%, and about 51%, respectively.

In some embodiments, the lipid nanoparticle comprises DMG-PEG2500, ionizable lipids, DSPC, cholesterol, and a stabilizer.

crRNAs

Disclosed herein, in some embodiments, are lipid nanoparticles comprising nucleic acids encoding for mosaic crNRA sequences for the treatment and prevention of HIV infections. In certain embodiments, the crRNA sequences bind to a DNA sequence within an HIV genome (e.g., HIV-1 or HIV-2).

In some embodiments, the crRNAs are “mosaic crRNAs.” In some embodiments, the mosaic crRNA is constructed from a multiple sequence alignment of separate HIV viral strains, for example separate HIV-1 or HIV-2 strains. In some embodiments, the target sequence of the mosaic crRNA is a theoretical composite of an HIV-1 or HIV-2 DNA sequences, for example sequences that retain a high (≥ 50%) or low (<50%) levels of conservation across isolated HIV strains.

HIV-1 and HIV-2 are two distinct viruses. HIV-1 is the most common HIV virus. HIV-2 occurs in a much smaller number of individual, mostly in individuals found in West Africa. In the U.S., HIV-2 makes up only 0.01% of all HIV cases. The 10 kilobase pair (kb) genome of HIV-1 encodes 3 structural (gag, pol, and env) polyproteins and 6 non-structural (tat, rev, vif, vpu, vpr, and nef) proteins from 3 overlapping alternate reading frames.

HIV-1 has four groups. Group M (Major) accounts for nearly 90% of all HIV-1 cases. HIV-1, group M has nine named strains: A, B, C, D, F, G, H, J, and K. Additionally, Different subtypes can combine genetic material to form a hybrid virus, known as a ‘circulating recombinant form’ (CRFs). HIV-1, group M, strain B strain is the most common strain of HIV in the U.S. Worldwide, the most common HIV strain is HIV-1, group M, strain C. HIV-1 has three additional groups —groups N, O, and P.

In some embodiments, a mosaic crRNA is constructed from a multiple sequence alignment of two or more HIV-1, group M strains selected from: A, B, C, D, F, G, H, J, and K.

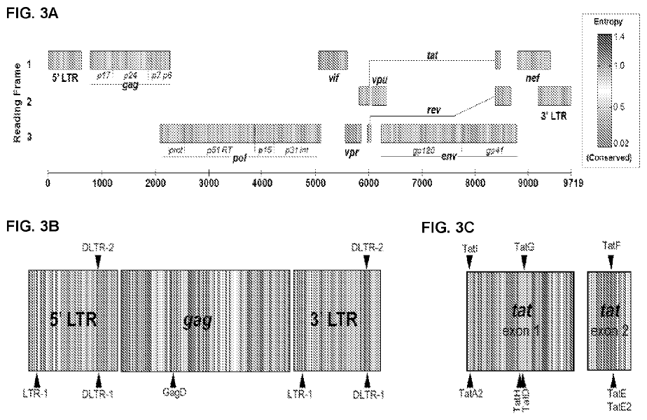

To construct mosaic crRNAs, a consensus HIV sequence can be created. The consensus sequence is based on the most recent alignment for the fullest spectrum of HIV-1 sequences, for example using the Los Alamos National Laboratory database for HIV sequence (hiv.lanl.gov). The Los Alamos database contains 4004 variant sequences. FIG. 3 summarizes the tat locus of all the 4004 sequences: the height of the letters corresponds to percentage of sequences that has that nucleotide in that specific location. For example the first position in FIG. 3 (location 5831 in the HXB2 reference genome) is an A—most of the sequences of the 4004 variants at location 5831 had an A. From all available sequences, a consensus sequence can be generated. Each nucleotide of the consensus sequence can be determined based on being present on most of the sequences, for example is at least 50% of sequences.

In some embodiments, a mosaic crRNA disclosed herein binds to a plurality of nucleic acids of an HIV-1 gene selected from the group consisting of: tat, rev, env-gp41, gag-p1, gag-p6, vif, vpr, vpu, and nef. In some embodiments, a mosaic crRNA disclosed herein binds to a plurality of nucleic acids of a gene encoding an HIV-1 protein selected from the group consisting of: Tat, Rev, Env-gp41, Gag-p1, Gag-p6, Vif, Vpr, Vpu, and Nef.

In some embodiments, a mosaic crRNA disclosed herein targets a consensus sequence derived from over 4000 HIV strains in a non-structural multiexon region. In some embodiments, the mosaic crRNA sequence is adjacent to an appropriate PAM sequence. In some embodiments, the mosaic crRNA sequence is adjacent to a S. pyogenes (spCas9) PAM sequence (NGG). In some embodiments, the mosaic crRNA sequence is adjacent to a S. aureus Cas9 (saCas9) PAM sequence (NNGRRT or NGRRN). PAMs for various Cas enzymes are described in Table 1 below, where “N” can be any nucleotide base.

TABLE 1

| CRISPR | | PAM Sequence |
|---|---|---|
| Nucleases | Organism Isolated From | (5′ to 3′) |
| SpCas9 | Streptococcus pyogenes | NGG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| LbCpf1 | Lachnospiraceae bacterium | TTTV |
| AsCpf1 | Acidaminococcus sp. | TTTV |

Advantages of the mosaic multiexon cleavage strategy are threefold. (1) First, mosaic crRNAs targeting multiexon regions superiorly reduce viral replication compared to crRNAs targeting LTR promoter DNA and single gene encoding proviral DNA as a result of CRISPR-Cas9 cleavage rather than excision. (2) Second, mosaic crRNAs retain broader coverage against transmitted founder HIV-1 strains compared to conventional CRISPR-Cas9 crRNAs designed against routinely tested laboratory strains of HIV. (3) Third, crRNAs targeting multiexon or regulatory regions display lower likelihood of generating CRISPR-resistant escape mutants.

In some embodiments, a mosaic crRNA disclosed herein binds to a plurality of nucleic acids of an overlapping exon. In some embodiments, the overlapping exon is part of a nucleic acid sequence of at least two HIV-1 genes selected from the group consisting of: tat, rev, env-gp41, gag-p1, gag-p6, vif, vpr, vpu, and nef. In some embodiments, the overlapping exon is part of a nucleic acid sequence of at least three HIV-1 genes selected from the group consisting of: tat, rev, env-gp41, gag-p1, gag-p6, vif, vpr, vpu, and nef. In some embodiments, In some embodiments, the overlapping exon is part of a nucleic acid sequence of HIV-1 genes tat, rev, and env.

In some embodiments, a mosaic crRNA disclosed herein binds to a plurality of nucleic acids of a HIV-1 sequence (HXB2, complete genome; HIV1/HTLV-III/LAV reference genome: GenBank: K03455.1) selected from: tat (exon 1, nucleic acids 5831-6045; exon 2, nucleic acids 8379-8469), rev (exon 1, nucleic acids 5970-6045; or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417).

In some embodiments, the mosaic crRNA is selected from a crRNA of Table 2 below:

TABLE 2

| crRNA | Target DNA Complementary | |
|---|---|---|
| Name | Sequence (5′→3′) | crRNA Seed Sequence (5′→3′) |
| TatA₂ | TAGATCCTAACCTAGAGCCC | UAGAUCCUAACCUAGAGCCC (SEQ |
| | (SEQ ID NO: 9) | ID NO. 1) |
| TatD | TCTCCTATGGCAGGAAGAAG | UCUCCUAUGGCAGGAAGAAG (SEQ |
| | (SEQ ID NO: 10) | ID NO: 2) |
| TatE | GAAGGAATCGAAGAAGAAGG | GAAGGAAUCGAAGAAGAAGG (SEQ |
| | (SEQ ID NO: 11) | ID NO: 3) |
| TatE₂ | GAAAGAATCGAAGAAGGAGG | GAAAGAAUCGAAGAAGGAGG (SEQ |
| | (SEQ ID NO: 12) | ID NO: 4) |
| TatF | CCGATTCCTTCGGGCCTGTC | CCGAUUCCUUCGGGCCUGUC (SEQ |
| | (SEQ ID NO: 13) | ID NO: 5) |
| TatG | TCTCCGCTTCTTCCTGCCAT | UCUCCGCUUCUUCCUGCCAU (SEQ |
| | (SEQ ID NO: 14) | ID NO: 6) |
| TatH | GCTTAGGCATCTCCTATGGC | GCUUAGGCAUCUCCUAUGGC (SEQ |
| | (SEQ ID NO: 15) | ID NO: 7) |
| TatI | GGCTCTAGGTTAGGATCTAC | GGCUCUAGGUUAGGAUCUAC (SEQ |
| | (SEQ ID NO: 16) | ID NO: 8) |

In some embodiments, the mosaic crRNA is TatA2-UAGAUCCUAACCUAGAGCCC (SEQ ID NO. 1). In some embodiments, the mosaic crRNA is TatD-UCUCCUAUGGCAGGAAGAAG (SEQ ID NO: 2). In some embodiments, the mosaic crRNA is TatE-GAAGGAAUCGAAGAAGAAGG (SEQ ID NO: 3). In some embodiments, the mosaic crRNA is TatE2-GAAAGAAUCGAAGAAGGAGG (SEQ ID NO: 4). In some embodiments, the mosaic crRNA is TatF—CCGAUUCCUUCGGGCCUGUC (SEQ ID NO: 5). In some embodiments, the mosaic crRNA is TatG-UCUCCGCUUCUUCCUGCCAU (SEQ ID NO: 6). In some embodiments, the mosaic crRNA is TatH-GCUUAGGCAUCUCCUAUGGC (SEQ ID NO: 7). In some embodiments, the mosaic crRNA is TatI-GGCUCUAGGUUAGGAUCUAC (SEQ ID NO: 8).

In some embodiments, the mosaic crRNA is 80%, 85%, 90%, or 95% identical to TatA2-UAGAUCCUAACCUAGAGCCC (SEQ ID NO. 1). In some embodiments, the mosaic crRNA is 80%, 85%, 90%, or 95% identical to TatD-UCUCCUAUGGCAGGAAGAAG (SEQ ID NO: 2). In some embodiments, the mosaic crRNA is 80%, 85%, 90%, or 95% identical to TatE-GAAGGAAUCGAAGAAGAAGG (SEQ ID NO: 3). In some embodiments, the mosaic crRNA is 80%, 85%, 90%, or 95% identical to TatE2-GAAAGAAUCGAAGAAGGAGG (SEQ ID NO: 4). In some embodiments, the mosaic crRNA is 80%, 85%, 90%, or 95% identical to TatF—CCGAUUCCUUCGGGCCUGUC (SEQ ID NO: 5). In some embodiments, the mosaic crRNA is 80%, 85%, 90%, or 95% identical to TatG-UCUCCGCUUCUUCCUGCCAU (SEQ ID NO: 6). In some embodiments, the mosaic crRNA is 80%, 85%, 90%, or 95% identical to TatH-GCUUAGGCAUCUCCUAUGGC (SEQ ID NO: 7). In some embodiments, the mosaic crRNA is 80%, 85%, 90%, or 95% identical to TatI-GGCUCUAGGUUAGGAUCUAC (SEQ ID NO: 8).

In some embodiments, a mosaic crRNA disclosed herein reduces HIV-1 replication by at least 50%. In some embodiments, a mosaic crRNA disclosed herein reduces HIV-1 replication by at least 60%, 65%, 70%, 75%, 80%, 85%, 90%, 95%, or 99%. In some embodiments, TatD reduces HIV-1 replication by at least 54%. In some embodiments, TatE reduces HIV-1 replication by 76%. In some embodiments, co-administration of TatD and TatE (TatDE) reduces HIV-1 replication by an average of 82% in 7 strains, including 6 clade B transmitted founder strains.

In some embodiments, a mosaic crRNA disclosed herein is effective against at least 50% of HIV-1 strains. In some embodiments, a mosaic crRNA disclosed herein is effective against at least 60%, 65%, 70%, 75%, 80%, 85%, 90%, 95%, or 99% of HIV-1 strains. In some embodiments, TatDE therapy is effective against at least 62% of all HIV-1 strains.

In some embodiments, a crRNA disclosed herein is operable with any suitable Cas enzyme. In some embodiments, a crRNA disclosed herein is operable with a Cas enzyme selected from the group consisting of: Cas9, CasPhi (Cas @), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10. Csf1, Csn2, Cas4, C2c1, C2c3, Cas12a (Cpf1), Cas12b, Cas12e, Cas13a, Cas13, Cas13c, and Cas13d. In some embodiments, a crRNA disclosed herein is operable with Cas9.

In some embodiments, a crRNA disclosed herein is part of a single guide RNA (“sgRNA”) sequence wherein the sgRNA sequence comprises the crRNA sequences and a tracrRNA sequence. Any suitable tracrRNA sequence is contemplated for use with a sgRNA disclosed herein. In some embodiments, the sgRNA comprises TatA2 and a tracrRNA sequence. In some embodiments, the sgRNA comprises TatD and a tracrRNA sequence. In some embodiments, the sgRNA comprises TatE and a tracrRNA sequence. In some embodiments, the sgRNA comprises TatE2 and a tracrRNA sequence. In some embodiments, the sgRNA comprises TatF and a tracrRNA sequence. In some embodiments, the sgRNA comprises TatG and a tracrRNA sequence. In some embodiments, the sgRNA comprises TatH and a tracrRNA sequence. In some embodiments, the sgRNA comprises TatI and a tracrRNA sequence.

In some embodiments, the crRNA sequence is a DNA sequence (such as single- or double stranded linear sequences: or plasmid DNA), an RNA sequence, or a recombinantly expressed crRNA/protein fusion (such as ribonucleoprotein (RNP)). In some embodiments, the DNA or RNA sequence comprising the crRNA sequence further comprises a tracrRNA sequence (e.g., a sgRNA sequence) and/or a sequence encoding a Cas9 enzyme.

CRISPR-Cas9) based therapeutics include but are not limited to CRISPR-Cas9 ribonucleoprotein (RNPs), guide RNAs and/or crRNAs that target or are complementary to one or more HIV-1 genes including but not limited to tat, rev, env-gp41, gag-p1, gag-p6, vif, vpr, vpu, and nef, and plasmids or other constructs containing the guide RNAs and/or crRNAs. In some embodiments, the guide RNAs and/or crRNAs include but are not limited to TatD, TatH, TatE, TatE2, TatA2, TatG, TatF, and/or combinations thereof, and/or plasmids containing TatD, TatH, TatE, TatE2, TatA2, TatG, TatF and/or combinations thereof or RNPs containing TatD, TatH, TatE, TatE2, TatA2, TatG, TatF, and/or combinations thereof as described in PCT/US2021/021246 (incorporated by reference herein) and/or mRNAs containing TatD, TatH, TatE, TatE2, TatA2, TatG, TatF and/or combinations thereof. In some embodiments the therapeutic agent is a combination of TatD and TatE guide RNAs and/or crRNAs, plasmids containing TatD and TatE guide RNAs or crRNAs, and/or RNPs containing TatD and TatE guide RNAs and/or crRNAs (the combination of TatD and TatE may be referred to as TatDE), or mRNAs containing TatD and TatE guide RNAs and/or crRNAs. In some embodiments, the CRISPR-Cas9 base therapeutic is encapsulated by the cationic lipid, which is in turn encapsulated by the remaining lipids (such as the zwitterionic lipid, the PEG-lipid conjugates, and the cholesterol). In some embodiments, the crRNA loaded into the lipid nanoparticle is selected from SEQ ID NO: 1-8. In some embodiments the crRNA loaded into the lipid nanoparticle is crRNA encoding for TatDE. In some embodiments the crRNA loaded into the lipid nanoparticle is selected from SEQ ID NO: 2 and SEQ ID NO: 3. In some embodiments, the crRNA sequence is encoded in a vector. In some embodiments the crRNA is a mRNA.

In some embodiments, a crRNA disclosed here (any of TatA₂, TatD, TatE, TatE₂, TatF, TatG, TatH, or TatI) or sgRNA disclosed herein (any of TatA₂/tracrRNA, TatD/tracrRNA, TatE/tracrRNA, TatE₂/tracrRNA, TatF/tracrRNA, TatG/tracrRNA, TatH/tracrRNA, or TatI/tracrRNA) is formulated as a lipid nanoparticle (LNP). A LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm.

In some embodiments, the composition comprises: TatD and TatH (TatD/H). In some embodiments, the composition comprises: TatD and TatE (TatD/E). In some embodiments, the composition comprises: TatE and TatH (TatE/H). In some embodiments, the composition comprises: TatD and TatA2 (TatA2/D).

In some embodiments, the lipid nanoparticle composition comprises: TatD/tracrRNA and TatH/tracrRNA. In some embodiments, the lipid nanoparticle composition comprises: TatD/tracrRNA and TatE/tracrRNA. In some embodiments, the lipid nanoparticle composition comprises: TatE/tracrRNA and TatH/tracrRNA. In some embodiments, the lipid nanoparticle composition comprises: TatD/tracrRNA and TatA2/tracrRNA.

In some embodiments, a crRNA disclosed here (any of TatA2, TatD, TatE, TatE2, TatF, TatG, TatH, or TatI) or sgRNA disclosed herein (any of TatA2/tracrRNA. TatD/tracrRNA, TatE/tracrRNA, TatE2/tracrRNA, TatF/tracrRNA, TatG/tracrRNA, TatH/tracrRNA, or TatI/tracrRNA) is part of a vector. In some embodiments, the Cas enzyme is part of a vector. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of the same vector. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of separate vectors.

In some embodiments, a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) is part of a mRNA. In some embodiments, the Cas enzyme is part of a mRNA. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of the same mRNA. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of separate mRNAs.

In some embodiments, a crRNA disclosed here (any of TatA2, TatD, TatE, TatE2, TatF, TatG, TatH, or TatI) or sgRNA disclosed herein (any of TatA2/tracrRNA. TatD/tracrRNA, TatE/tracrRNA, TatE2/tracrRNA, TatF/tracrRNA, TatG/tracrRNA, TatH/tracrRNA, or TatI/tracrRNA) is enveloped in a LNP. In some embodiments, the Cas enzyme enveloped in a LNP. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA. TatG/tracrRNA, TatH/tracrRNA, or TatI/tracrRNA) and the Cas enzyme are enveloped in the same LNP. In some embodiments, the crRNA disclosed here (any of TatA2, TatD, TatE, TatE2, TatF, TatG, TatH, or TatI) or sgRNA disclosed herein (any of TatA2/tracrRNA. TatD/tracrRNA, TatE/tracrRNA, TatE2/tracrRNA, TatF/tracrRNA, TatG/tracrRNA, TatH/tracrRNA, or TatI/tracrRNA) and the Cas enzyme are enveloped in separate LNPs.

In some embodiments, the lipid nanoparticle comprises a mixture of DSPE-PEG2000, DOPE, Cholesterol, DMG-PEG, and DOTAP with molar percent of 10.83%, 11.25%, 24.06%, 2.78%, and 51.07%, respectively and encapsulates CRISPR HIV-1 TatDE plasmid.

In some embodiments, the lipid nanoparticle comprises a mixture of DMG-PEG2500, ionizable lipids, DSPC, cholesterol, and a stabilizer and encapsulates CRISPR HIV-1 TatDE mRNA.

### Diagnostics

Disclosed herein, in certain embodiments, are diagnostic agents comprising the lipid nanoparticles of the present invention. In some embodiments, the diagnostic agent is selected from: MRI contrast agents, fluorescent dyes, and nuclear medicine agents (e.g. PET or SPECT radioisotopes). In some embodiments, the diagnostic agent is a radiolabeled europium doped cobalt ferrite nanoparticle (177Lu/89ZrCFEu nanoparticles). In some embodiments, Lutetium-177 or Zirconium-89 (177Lu or 89Zr)-radiolabeled CFEu nanoparticles are produced for bioimaging tests using a modified solvothermal technique, where 177Lu or 89Zr label was made containing iron (III) acetylacetonate, cobalt (II) acetylacetonate and europium (III) nitrate pentahydrate. These are dissolved by sonication in benzyl alcohol (as the solvent) in the presence of reducing and stabilizing agents 1,2-hexadecanediol, oleic acid and oleamine. Upon nucleation reaction in a hydrothermal autoclave reactor, the nanoparticles were purified by ethanol and centrifugations. These ¹⁷⁷Lu⁸⁹Zr-labeled CFEu nanoparticles can then be loaded into the lipid nanoparticles with or without a therapeutic agent.

Also disclosed herein, in certain embodiments, are lipid nanoparticles further comprising a diagnostic agent. In some embodiments, the diagnostic agent is a MRI contrast agent, a fluorescent dye, or a nuclear medicine agent. In some embodiments, the diagnostic agent is a radiolabeled europium doped cobalt ferrite nanoparticle (177Lu/89ZrCFEu nanoparticles).

In some embodiments, provided herein are methods of use of the lipid nanoparticles further comprising a diagnostic agent, comprising administering the lipid nanoparticles to an individual in need thereof. In some embodiments, disclosed herein is a method of diagnosing an individual in need of HIV-1 therapy, comprising administering to the individual the lipid nanoparticle further comprising a diagnostic agent or a pharmaceutical composition comprising a lipid nanoparticle further comprising a diagnostic agent and a pharmaceutically acceptable excipient.

### Pharmaceutical Compositions

Disclosed herein, in certain embodiments, are pharmaceutical compositions comprising a plurality of lipids and a CRISPR nucleic acid complementary to a HIV-1 gene, and a pharmaceutically acceptable excipient. In some embodiments, the pharmaceutical composition comprises (a) a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA), and (b) a pharmaceutically acceptable excipient. In some embodiments, the composition further comprises a Cas enzyme.

In some embodiments, the pharmaceutical composition comprises: TatD and TatH (TatD/H). In some embodiments, the pharmaceutical composition comprises: TatD and TatE (TatD/E). In some embodiments, the pharmaceutical composition comprises: TatE and TatH (TatE/H). In some embodiments, the pharmaceutical composition comprises: TatD and TatA2 (TatA2/D).

In some embodiments, the pharmaceutical composition comprises: TatD/tracrRNA and TatH/tracrRNA. In some embodiments, the pharmaceutical composition comprises: TatD/tracrRNA and TatE/tracrRNA. In some embodiments, the pharmaceutical composition comprises: TatE/tracrRNA and TatH/tracrRNA. In some embodiments, the pharmaceutical composition comprises: TatD/tracrRNA and TatA2/tracrRNA.

In some embodiments, a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) is part of a viral vector. In some embodiments, the Cas enzyme is part of a viral vector. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of the same viral vector. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of separate viral vectors.

In some embodiments, the pharmaceutically acceptable excipient is a carrier, solvent, stabilizer, adjuvant, diluent, etc., depending upon the particular mode of administration and dosage form.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, cellulose, dextrin).

In some embodiments, the composition has a physiologically compatible pH (e.g., a range from a pH of about 3 to a pH of about 11, about pH 3 to about pH 7, depending on the formulation and route of administration). In some cases, the pH is from about pH 5.0 to about pH 8.

In some embodiments, the composition further comprises a second active ingredient useful in the treatment or prevention of bacterial growth (for example and without limitation, anti-bacterial or anti-microbial agents).

### Methods of Use

Disclosed herein, in certain embodiments, are methods of treating an HIV-1 infection in an individual in need thereof. Further disclosed herein, in certain embodiments, are methods of preventing an HIV-1 infection in an individual in need thereof. Additionally, disclosed herein, in certain embodiments, are methods of preventing transmission of an HIV-1 virus from one individual to another (for example, from a pregnant woman to a child, for example during birth or breast feeding).

In some embodiments, the methods comprise administering to an individual a lipid nanoparticle comprising a plurality of lipids and a CRISPR nucleic acid disclosed here. In some embodiments, the crRNA comprises any of TatA2, TatD, TatE, TatE2, TatF, TatG, TatH, or TatI. In some embodiments, the methods comprise administering to an individual any lipid nanoparticle comprising a combination of a crRNA disclosed here. In some embodiments, the method comprises administering to the individual: TatD and TatH (TatD/H). In some embodiments, the method comprises administering to the individual: TatD and TatE (TatD/E). In some embodiments, the method comprises administering to the individual: TatE and TatH (TatE/H). In some embodiments, the method comprises administering to the individual: TatD and TatA2 (TatA2/D).

In some embodiments, the methods comprise administering to an individual any sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA). In some embodiments, the methods comprise administering to an individual any combination of a sgRNA disclosed here. In some embodiments, the method comprises administering to the individual: TatD/tracrRNA and TatH/tracrRNA. In some embodiments, the method comprises administering to the individual: TatD/tracrRNA and TatE/tracrRNA. In some embodiments, the method comprises administering to the individual: TatE/tracrRNA and TatH/tracrRNA. In some embodiments, the method comprises administering to the individual: TatD/tracrRNA and TatA2/tracrRNA.

In some embodiments, the method dysregulates virion production from a latent proviral DNA or impede integration of reverse-transcribed proviral DNA. In some embodiments, the crRNA is a mosaic crRNA. In some embodiments, the crRNA binds to a plurality of nucleic acids of an overlapping exon of at least two HIV-1 genes. In some embodiments, the crRNA binds to a plurality of nucleic acids of an overlapping exon of at least three HIV-1 genes.

Further disclosed herein, in certain embodiments, are methods of excising all or a portion of an HIV genome in an HIV infected cell of an individual. In some embodiments, the method comprises administering to the individual a lipid nanoparticle comprising a first crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) that binds to a first HIV sequence and a second a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) that binds to a second HIV sequence, provided that the first crRNA or sgRNA and the second crRNA or sgRNA are different crRNAs or sgRNAs. In some embodiments, at least one of the first crRNA and the second crRNA is a mosaic crRNA. In some embodiments, at least one of the first crRNA and the second crRNA binds to a plurality of nucleic acids of an overlapping exon of at least two HIV-1 genes. In some embodiments, at least one of the first crRNA and the second crRNA binds to a plurality of nucleic acids of an overlapping exon of at least three HIV-1 genes.

A pharmaceutical composition disclosed herein is administered by any appropriate route that results in effective treatment in the subject. In some embodiments, a pharmaceutical composition disclosed herein is administered systemically. In some embodiments, a pharmaceutical composition disclosed herein is administered locally. The pharmaceutical composition is administered via a route such as, but not limited to, enteral, gastroenteral, oral, transdermal, subcutaneous, nasal, intravenous, intravenous bolus, intravenous drip, intraarterial, intramuscular, transmucosal, insufflation, sublingual, buccal, conjunctival, cutaneous. Modes of administration include injection, infusion, instillation, and/or ingestion. “Injection” includes, without limitation, intravenous, intramuscular, intra-arterial, intrathecal, intraventricular, intradermal, intraperitoneal, transtracheal, and subcutaneous. In some examples, the route is intravenous.

The nonviral lipid nanoparticle (LNP) delivery system were made based on their ease of manufacture, limited immune responses, larger payloads, and ease of design. These may be produced by thin-film hydration followed by dialysis cassette purification and the use of microfluidic channels. The lipid nanoparticles of the present invention can be used for the treatment, prevention, and or disease elimination. In some embodiments, the disease is HIV. The lipid nanoparticles of the present invention may be administered to a patient and may be conveniently formulated for administration with any pharmaceutically acceptable carrier(s).

The lipid nanoparticles of the present invention may be administered by any method. Methods of administration include but are not limited to parenterally, subcutaneously, orally, topically, pulmonary, rectally, vaginally, intravenously, intraperitoneally, intrathecally, intracerbrally, epidurally, intramuscularly or intradermally.

### EXAMPLES

### Example 1 Preparation of LNPs

Microfluidic techniques were used to synthesize LNPs containing either CRISPR-Cas9 plasmids or RNP (targeting LTRgag, CCR5 or TatDE). LNPs included cholesterol, PEG-lipids (DSPE-PEG2000, and DMG-PEG), zwitterionic lipid (DOPE), and cationic lipid (DOTAP). Lipid mixtures, prodrugs, and radiolabeled nanoparticles were passed through microfluidic microchannels under controlled pressure and flow rate to prepare radiolabeled CRISPR-Cas9) plasmid/RNP LNPs (CRISPR-Cas9@¹⁷⁷Lu/⁸⁹ZrCFEu).

First, correct PEG lipids were identified as DSPE-PEG2000 (1,2-distearoyl-sn-glycero-3-phosphoethanolamine-N-amino (polyethylene glycol)-2000) and DMG-PEG (1,2-dimyristoyl-rac-glycero-3-methoxypolyethylene glycol-2000). Zwitterionic lipid as DOPE (1,2-dioleoyl-sn-glycero-3-phosphoethanolamine), anionic lipid as 12:0 PG (1,2-dilauroyl-sn-glycero-3-phospho-(1′-rac-glycerol) or cationic lipid as DOTAP (1,2-dioleoyl-3-trimethylammonium-propane) and cholesterol. Molar ratios of lipid mixture were selected for DSPE-PEG2000 (11 mole %), DOPE (11.25 mole %), DMG-PEG (2.780) mole %), DOTAP or 12:0 PG (51.07 mole %) and Cholesterol (24.06mole %).

Second, lipid mixtures were prepared in the molecular biology grade ethanol to ensure a homogeneous mixture in the ethanol at 60° C.

Third, the solvent was removed to yield a lipid film by rotary evaporation in a round bottom flask and vacuumed dry overnight.

Following, TatDE plasmid or RNPs cargo (1:1 weight ratio of lipid to the gene) will be suspended in ultrapure sterile water, TatDE plasmid suspension will be added to lipids film and liposomal suspension were form with rotation under warm (˜ 30 min) water or until complete form liposomal suspension. DNA loaded liposomal suspension was placed into a dialysis bag (MW. 3.5-5 kDa)-Float-A-Lyzer®: Dialysis Device-Spectrum: A Repligen Brand). After an overnight dialysis liposomal suspension was made against 3000 ml ultrapure water at 10-15° C. under a fume hood and samples collected in Eppendorf tube and syringe filter (0.45 μM) (Milex-HP Hydrophilic). The filtrate was stored at 2° C. to 8° C. and particle size and polydispersity measured by dynamic light scattering (DLS) using a Malvern zeta sizer DLS instrument.

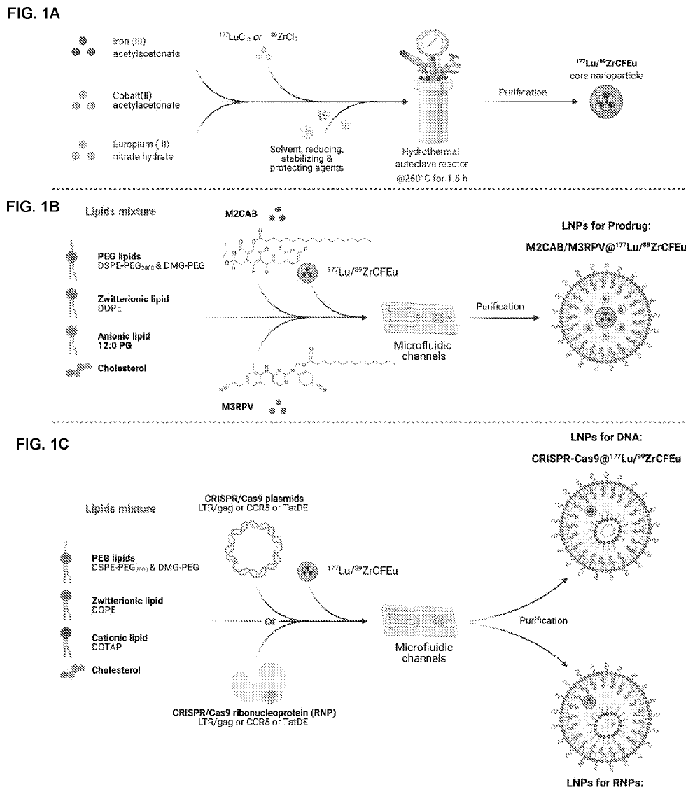

FIGS. 1A-C illustrates CRISPR-Cas9 nanoparticle synthesis. FIG. 1A shows an exemplary schematic for preparation of radiolabeled europium doped cobalt ferrite nanoparticles (¹⁷⁷Lu/⁸⁹ZrCFEu). The particles were manufactured by a modified solvothermal technique. Lutetium-177 or Zirconium-89 were made containing Iron (III) acetylacetonate, cobalt (II) acetylacetonate and europium (III) nitrate pentahydrate. The color graphical descriptions are as follow. Red spheres are iron; blue spheres are cobalt and pink spheres are europium. These are dissolved by sonication in benzyl alcohol (as the solvent) in the presence of reducing and stabilizing agents 1,2-hexadecanediol, oleic acid and oleamine. Upon nucleation reaction in a hydrothermal autoclave reactor, the nanoparticles were purified by ethanol and centrifugations. FIG. 1B shows an exemplary schematic for radiolabeled prodrug made in lipid nanoparticles (LNPs). Microfluidic techniques was used to synthesize LNPs containing the cabotegravir prodrug (M2CAB) and rilpivirine (M3RPV) with the bioimaging agent ¹⁷⁷Lu/⁸⁹ZrCFEu. LNP synthesis included cholesterol, PEG-lipids (DSPE-PEG2000, and DMG-PEG), zwitterionic lipid (DOPE), and anionic lipid (12:0 PG). Lipid mixtures, prodrugs, and radiolabeled nanoparticles were passed through microfluidic microchannels under controlled pressures and flow rates to prepare the radiolabeled prodrug lipid nanoparticles (M2CAB/M3RPV@¹⁷⁷Lu/⁸⁹ZrCFEu). The loaded LNPs were purified by dialysis. FIG. 1C shows an exemplary schematic for preparation of radiolabeled CRISPR-Cas9) plasmid and ribonucleoprotein (RNP) LNPs.

### Example 2 CRISPR HIV-1 TatDE LNPs

This example describes the production of CRISPR HIV-1 TatDE Plasmid LNPs and CRISPR HIV-1 TatDE mRNA LNPs.

For CRISPR HIV-1 TatDE plasmid LNPs, CRISPR HIV-1 TatDE plasmid was prepared and enveloped in a mixture of DSPE-PEG2000, DOPE, Cholesterol, DMG-PEG, and DOTAP with molar percent of 10.83%, 11.25%, 24.06%, 2.78%, and 51.07%, respectively.

For CRISPR HIV-1 TatDE mRNA LNPs, CRISPR mRNA for gRNA and spCas9 were prepared and enveloped in a mixture of DMG-PEG2500, ionizable lipids, DSPC, cholesterol, and a stabilizer.

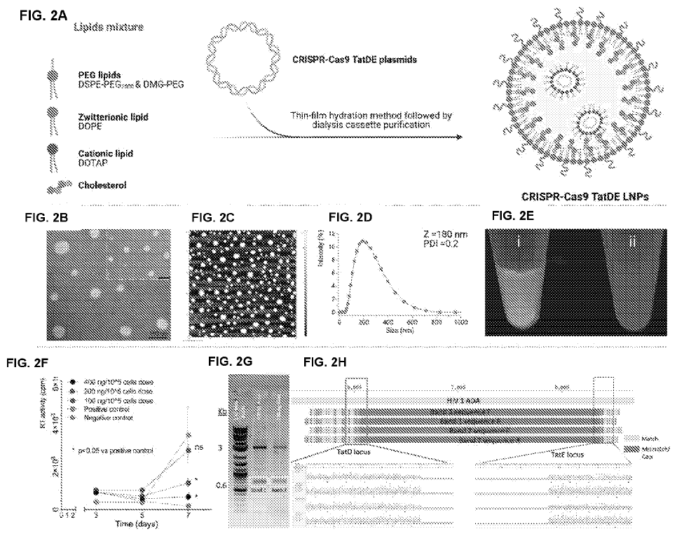

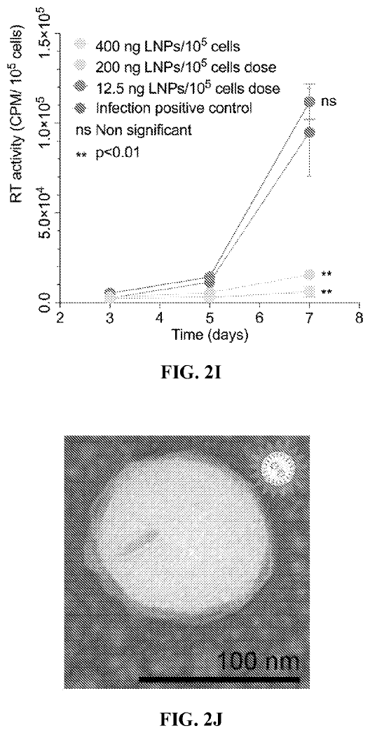

The LNPs were used for delivery of recombinant DNA (rDNA) or mRNA (ratio of gRNA:Cas 9 of 50:50) into mammalian cells (U1 cells and JLat 8.4 cells). Tested ratios 400 ng LNP/10\hat{ }5 cells, 200 ng LNPs/10\hat{ }5 cells, and 12.5 ng LNP/10\hat{ }5 cells. Results are shown in FIG. 2I.

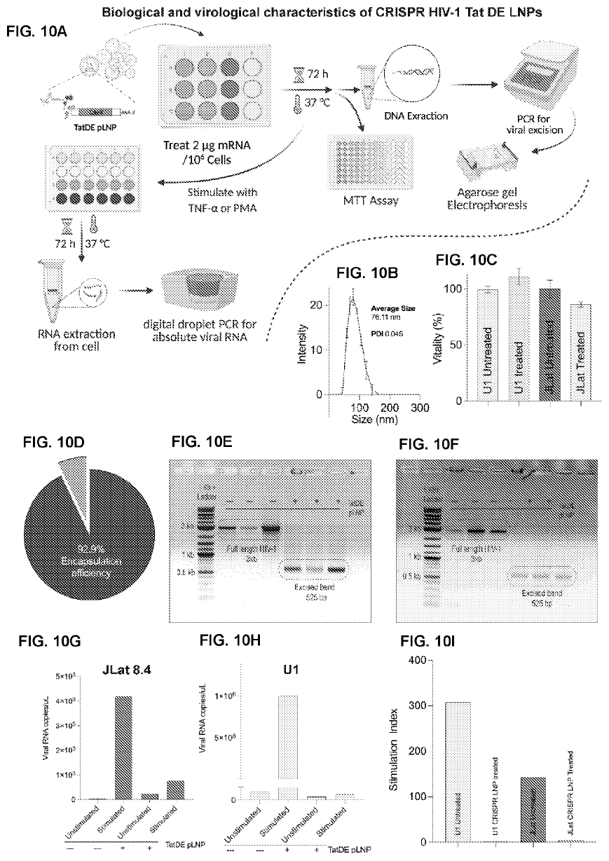

Using the LNPs to deliver HIV-1 CRISPR tatDE mRNA LNPs proved both sensitive and effective in eliminating more than 92% of integrated proviral DNA without evidence of cytotoxicities. Results are shown in FIGS. 10A-I.

FIGS. 2A-J illustrates the synthesis, characterization and antiretroviral activity of CRISPR-Cas9 lipid nanoparticles (LNPs) in primary human monocyte-derived macrophage (MDM). FIG. 2A shows exemplary process for CRISPR-Cas9 TatDE LNPs prepared by thin film hydration by mixing cholesterol, PEG-lipids (DSPE-PEG2000, and DMG-PEG), zwitterionic lipid (DOPE) and cationic lipid (DOTAP) with the CRISPR-Cas9 TatDE plasmid. The prepared mixture was dialyzed prior to virologic testing. FIG. 2B shows exemplary transmission electron microscopy (TEM) images of the CRISPR-Cas9 loaded LNPs. The scale bar is 100 nm. FIG. 2C shows exemplary atomic force microscopy (AFM) topographic images of the loaded LNPs demonstrate average height profiles. FIG. 2D shows an exemplary dynamic light scattering (DLS) graph that illustrates the size distribution of CRISPR-Cas9 loaded LNPs with an average size of 180 nm (PDI=0.2). FIG. 2E shows exemplary ethidium bromide (EtBr)-stained LNPs(i) fluorescing under ultraviolet (UV) excitation compared against unstained LNPs(ii). FIG. 2F shows RT activity over time for MDM treated with the CRISPR-Cas9 LNPs at a concentration of 100-400 ng TatDE particles/cell then challenged with HIV-1_{ADA} (macrophage tropic viral strain) at a multiplicity of infection (MOI) of 0.01 infectious viral particles/cell. HIV-1 infection was monitored by levels of reverse transcriptase (RT) activity reflective of progeny virions in culture fluids for a time period of 7 days. FIG. 2G shows exemplary image related to polymerase chain reaction (PCR) was performed in cell lysates followed by agarose gel electrophoresis. This confirmed protection was size of the “putative” virus-excised band. FIG. 2H shows the sized band was confirmed as the appropriate CRISPR-Cas9 excised subgenomic viral DNA by Sanger sequencing by the multiple alignment program for nucleotide sequences (MAFFT). FIG. 2I shows RT activity over time for MDM treated with the CRISPR-Cas9 LNPs at a concentration of 100-400 ng TatDE equivalent then challenged with HIV-1_{ADA} (macrophage tropic viral strain) at an MOI of 0.02. HIV-1 replication was monitored by RT activity in culture fluids as an indication of progeny virions for up to 7 days. Statistical significance was calculated using one way ANOVA for multiple comparisons (n=3) with Dunnets post hoc test. For 400 ng concentration. P=0.01; for 200 ng concentration P=0.04. PCR was performed in cell lysates using HIV-1 specific primers, followed by agarose gel electrophoresis. The excised band at ˜2.4 kb is highlighted in yellow confirms “putative” protection. FIG. 2J shows exemplary transmission electron microscopy images of the CRISPR-Cas9 loaded LNPs showed spherical morphology including a particle surface corona. The scale bar is 100 nm.

FIGS. 3A-C illustrates HIV CRISPR-Cas9 Mosaic gRNA Design. FIG. 3A shows nucleotide heterogeneity of 4004 annotated HIV-1 strains depicted in a heat-map form demonstrating entropic (blue) or conserved (red) loci in three reading frames. Prior reported gRNAs against LTR and gag regions were used as reference controls FIG. 3B shows designed gRNAs targeting mosaic HIV-1tat sequences, and FIG. 3C shows for antisense or sense sequences are shown by down or upward facing arrows, respectively.

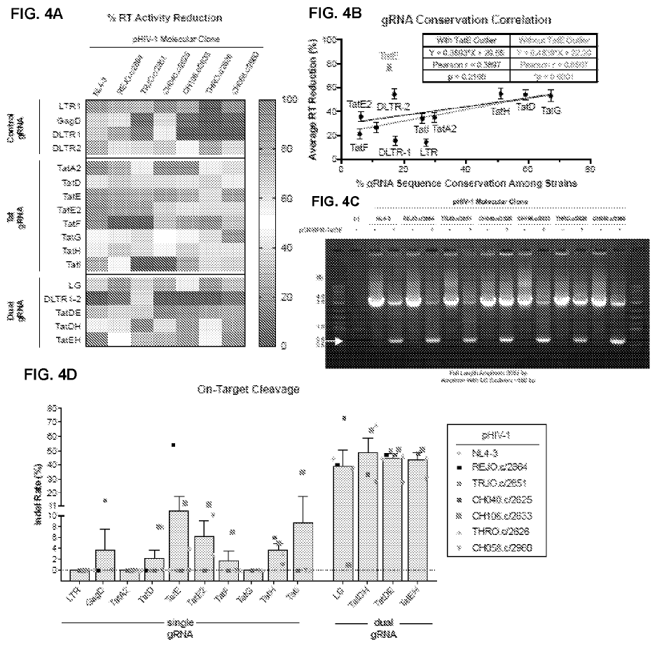

FIGS. 4A-D illustrates TatDE gRNAs Facilitate Multistrain HIV-1 Excision. FIG. 4A shows a gRNA library was screened against a panel of HIV-1 molecular clones by co-transfection into HEK293FT cells. Progeny virion production was measured by reverse transcriptase (RT) activity in culture fluids. FIG. 4B shows a Pearson correlation between gRNA target conservation among 4004 proviral DNA sequences and RT knockdown were assessed. FIG. 4C shows PCR tests were completed on DNA extracted from amplified untreated or CRISPR-TatDE plasmid-treated cells. The white arrow indicates the expected molecular size of the TatDE excision band. FIG. 4D shows PCR reaction contents were Sanger sequenced and evaluated in Inference of CRISPR Edits v2.0 (ICE. Synthego 2020) to visualize nucleotide editing in the PAM/protospacer regions. Data in (a-b, d) depict mean±standard error of the mean (SEM) from four independent experiments. Each of the experiments were performed in triplicate.

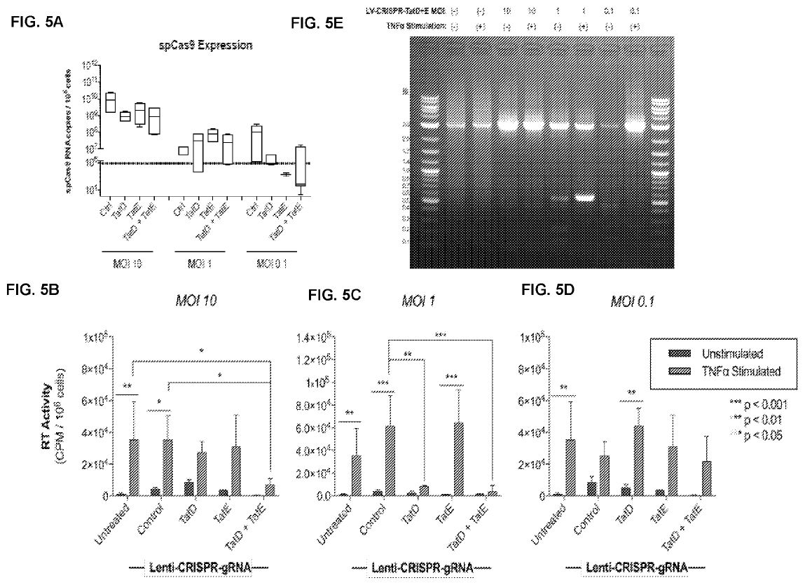

FIGS. 5A-E illustrates lentiviral TatDE CRISPR Inactivates Latent HIV-1. ACH2 T cells bearing a single copy of HIV-1 proviral DNA were transduced with lentivirus bearing a spCas9-gRNA transgene at multiplicities of infection (MOI) of 10, 1, or 0.1. After 72 hours, cells were stimulated with tumor necrosis factor alpha (TNFα. 15 ng/mL) for 72 hours. FIG. 5A shows spCas9 expression was measured by RT-qPCR. FIGS. 5B-D show RT activity recorded from culture supernatant fluids. FIG. 5E shows nested PCR for assayed proviral DNA excision wherein unedited amplicons are 2986 bp and CRISPR-edited amplicons are approximately 525 bp. These differences are dependent on insertion-deletion mutagenesis. The arrow indicates the expected molecular size in the presence of TatDE excision gRNAs. Significance was determined by a two-way ANOVA.

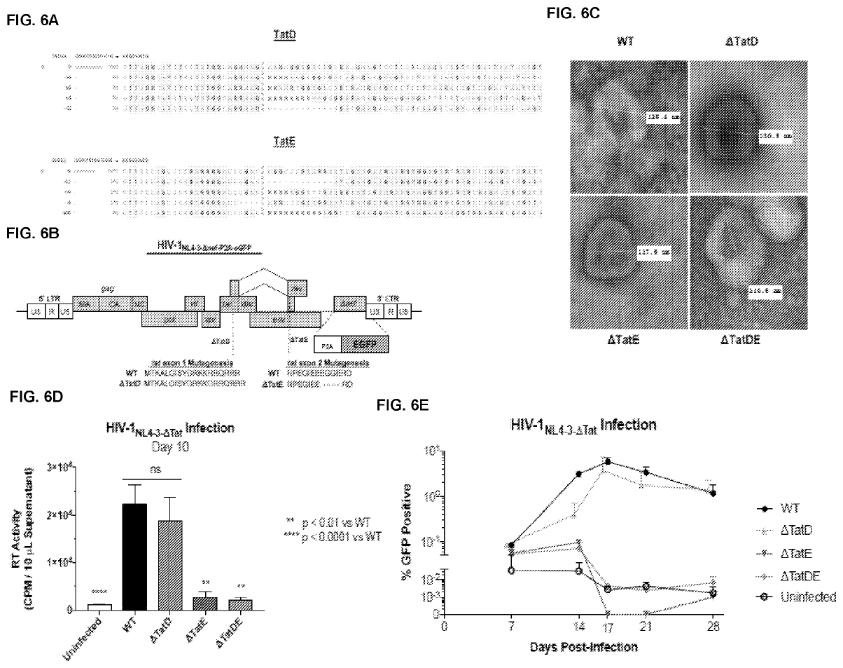

FIGS. 6A-E illustrates Exonic Disruption and HIV-1 Replicative Fitness. FIGS. 6A-B show insertion-deletion profiles among the generated gRNAs obtained through a co-transfection screen were assessed by the Synthego ICE v2.0 algorithm. The highest frequency insertions or deletions were selected for subsequent non-frameshift site-directed mutagenesis of the HIV-1_{NL4-3-Δnef-eGFP} encoding plasmid. FIG. 6C shows exemplary transmission electron micrographs of single- or dual-tat mutants are illustrated. Spherical diameter measurements were taken (inset). FIGS. 6D-E show CEMss CD4+ T cell lines were challenged with HIV-1_{NL4-3-Δtat-Δnef-eGFP} at an MOI of MOI 0.1 and assayed at defined time points for RT activity (FIG. 6D). Flow cytometry assay results for % GFP-positive cells are shown in FIG. 6E.

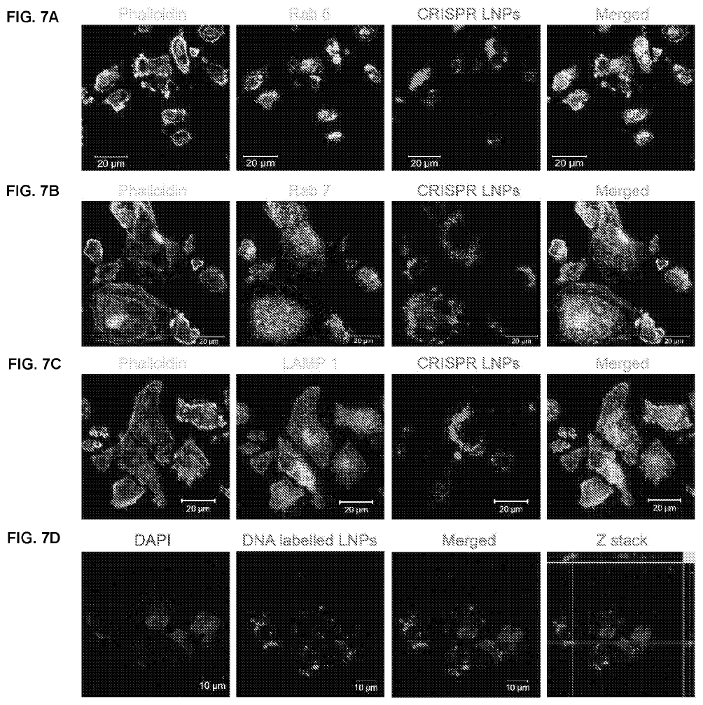

FIGS. 7A-D illustrates CRISPR LNPs cell trafficking. Rhodamine DHPE phospholipid tracked the locale of CRISPR LNPs in human MDMs. Confocal microscopy was employed 12 h after particle injection in the MDM cultures. Alexa-Fluor 488 (green) secondary antibody detected Rab 5. Rab7, or Lamp1 subcellular compartments. Phalloidin-iFluor 647 marked cell boundaries. The MDM nucleus was stained with DAPI (blue). Rhodamine DHPE phospholipid containing CRISPR-LNPs (red) colocalized with Rab5 (FIG. 7A) and Rab7 (green) (FIG. 7B). FIG. 7C shows no-colocalization was found between Lamp1 (green) and the nanoparticles (red). (FIG. 7D) TM-Rhodamine labeled px333DE was used for CRISPR LNPs to examine nuclear localization of the CRISPR payload present in the nucleus 12 h after treatment. Z-stack affirmed that the CRISPR reached the nucleus.

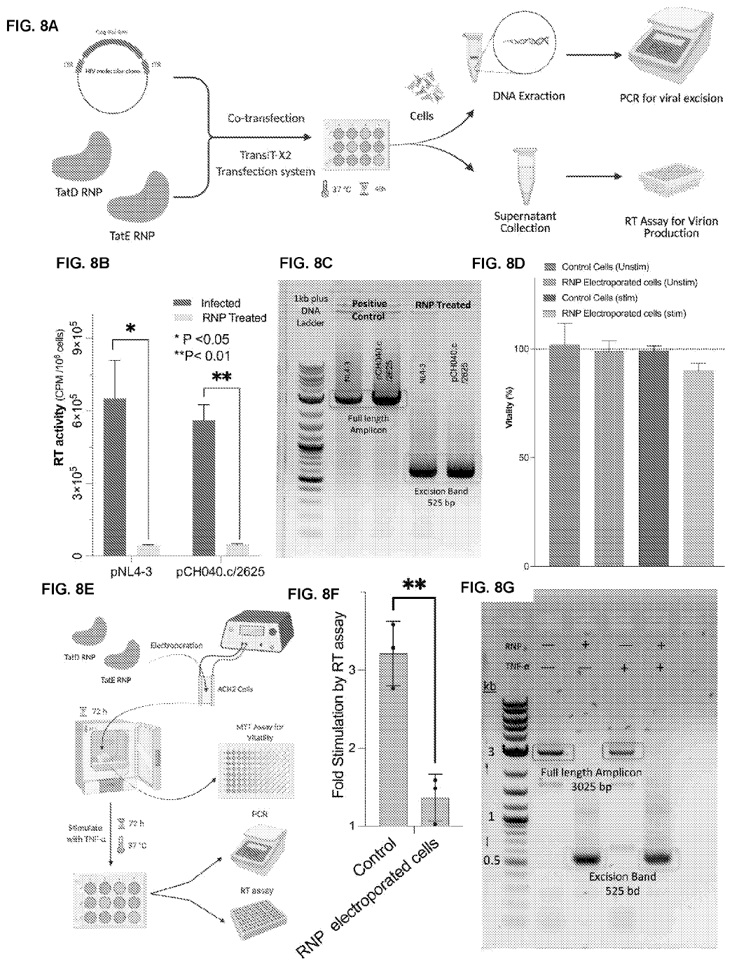

FIGS. 8A-G Illustrates HIV-1 RNP Delivery for Virus Editing. FIG. 8A shows TatD/TatE RNPs were assembled then co-transfected with two infectious HIV-1 molecular clones by TransIT-X2 transfection into HEK 293FT cells to determine Cas9 efficacy. FIG. 8B shows measurements in supernatants from transfected cells show that the HIV-1 RNP treatment reduces virion production to or around control levels. For NL4-3. P=0.02, for pCH040.c/2625 P=0.01 by unpaired t test compared to untreated infected control. FIG. 8C shows DNA PCR tests from the HIV-1 proviral clones show that all HIV-1 DNA was cleaved. FIG. 8D shows cell vitality MTT assay performed on the electroporated cells showed no significant change in cell viability. One-way ANOVA (n=3) with Dunnet's post-hoc test showed non-significant differences between different groups: P=0.54 and F=0.77. After 72 hours following electroporation. ACH2 cells were stimulated with TNF-α (15 ng/mL). FIG. 8E shows the efficacy of TatD/TatE RNPs were tested for viral excision in latent HIV-1 infected ACH2 cells. These cells carry a single copy of proviral DNA. RNPs were delivered to the ACH2 infected cells by electroporation. FIG. 8F shows tests for RT activity in culture fluids showed that the RNP treated ACH2s upon stimulation did not produce progeny virus. Untreated control cells showed significantly higher fold stimulation; P=0.01 by unpaired t-test. Then, viral excision was analyzed by DNA PCR. FIG. 8G shows PCR tests showed intact viral genome (3025 bp) in untreated controls, whereas full length HIV-1 proviral DNA was not detected in the treated groups. An expected 525 bp excised amplicon was readily seen in both stimulated and unstimulated RNP treated cells. Data points in FIG. 8B. FIG. 8D and FIG. 8F depict mean±SEM from biological triplicates.

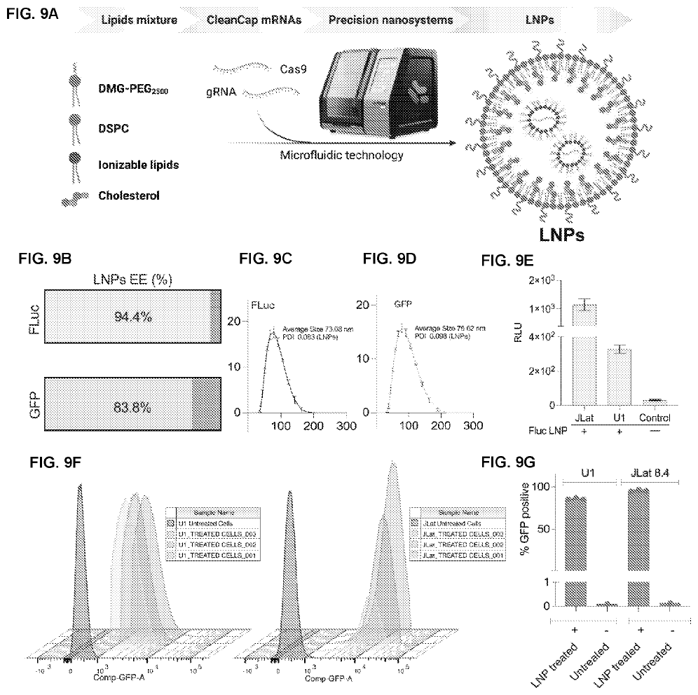

FIGS. 9A-G illustrates mRNA Loaded TatDE LNPs. FIG. 9A shows an exemplary schematic representation of the LNP components and the manufacturing process using non turbulent microfluidic mixing. FIG. 9B shows LNPs loaded with CleanCap Firefly luciferase (Fluc) mRNA and Dasher GFP mRNA showed high encapsulation efficiency of 94.4% and 83.8% quantified by the Ribogreen RNA assay kit. FIGS. 9C-D show Fluc and GFP LNPs had a very narrow size distribution with PDI of 0.083 and 0.098 respectively. They had an average diameter of 73.08 nm and 76.68 nm. U1 and JLat cells were treated with Fluc LNP at 2 μg mRNA equivalent/million cells dose. Forty eight hours following treatment cells were lysed and a luciferase assay was performed. FIG. 9E shows both cell lines show robust luminescence upon addition of Luciferase substrate to cell lysate confirming expression of luciferase delivered by LNP. FIG. 9F shows GFP LNP treatment to the cells shows high GFP expression in both U1 and JLat cell lines affirmed by shift of population from GFP dim to GFP positive cells. FIG. 9G shows eighty six and a half percent of the gated live singlet U1 cells were positive for GFP expression on an average and >98% of the gated live singlet JLat 8.4 cells showed GFP expression. Control cells showed less than 0.1% GFP expression. All the treatment experiments (GFP or FLuc) were performed in triplicates.

FIGS. 10A-I illustrates that LNP Cargos HIV-1 TatDE gRNA and Cas9 mRNA attenuate viral replication. CleanCap Cas9 mRNA, TatD and TatE sgRNA was combined and formulated using optimized lipid mix aided by microfluidic mixing to formulate TatDE plasmid LNP (pLNP). They were characterized and tested for anti-viral efficacy. FIG. 10A shows an exemplary schematic representation of antiviral efficacy and toxicity testing. FIG. 10B shows that TatDE pLNP show a narrower size distribution with a PDI of 0.045 and average diameter of 76.11 nm. FIG. 10D shows they also had a high encapsulation efficiency of 92.9%, TatDE pLNP was added to cells at 2 ug Cas9 mRNA equivalent per million of U1 or JLat 8.4 cells. FIG. 10C shows 72 hours post treatment cells were tested for LNP mediated toxicity using MTT vitality assay, TatDE pLNP was non-toxic for the U1 cells (˜100% vitality in treated group) and JLat cells (>85% vitality in treated group) compared to untreated controls. U1 and JLat 8.4 cells contain one or more integrated copy of viral genome in each cell. Cell genomic DNA were isolated and nested PCR was performed with HIV specific primers. Agarose gel electrophoresis of PCR product showed in both U1 cells (FIG. 10E) and JLat cells (FIG. 10F) full length HIV was present in untreated cells but not treated cells. Treated cells rather had an expected 525 bp excised fragment. PCR was ran from three independent samples. Subsequently treated cells were stimulated with 20 ng/ml TNF-α (JLat 8.4 cells) or 50 nM pMA (U1 cells). 72 hours post stimulation, cells were harvested, and RNA was extracted. FIGS. 10G-H show highly sensitive digital droplet PCR showed induction of HIV RNA production in untreated cells whereas in case of treated groups even after stimulation HIV RNA production was near baseline. FIG. 10I shows that stimulation causes approximately 100 (JLat) to 300 (U1) fold increase in RNA production in untreated cells but almost baseline level stimulation was seen in case of treated groups.

### Example 3 LNPs as Delivery System to Excise Integrated Latent Proviral DNA

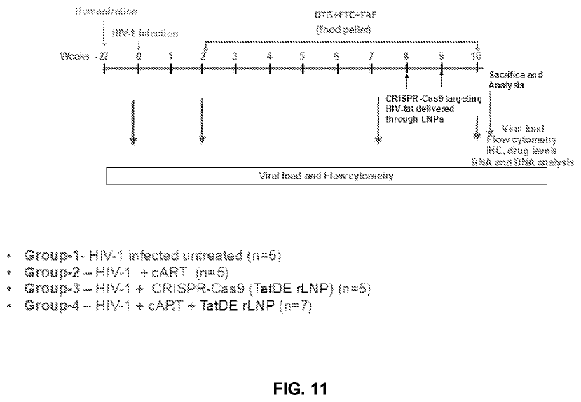

The following animal experiments affirm use of lipid nanoparticles (LNPs) as a delivery system to excise integrated latent proviral DNA in HIV-1 infected humanized mice. Editing efficiency CRISPR-Cas9 for HIV-1 elimination was performed by increasing the delivery efficiency of therapeutic gene payloads to infected tissues (FIG. 11). Viral strain diversity, offsite toxicity, transduction efficiencies, carrying capacity (greater than 4.7 kb of oligonucleotide payloads), and hepatoxicity were reduced by creating CRISPR-Cas9 guide RNAs (gRNAs) targeting conserved regions of multiple viral genes disrupt five HIV-1 exons (tat₁₋₂/rev₁₋₂/gp41) derived from a tat consensus sequence from 4004 HIV-1 strains (called TatDE), TatDE gRNA-Cas9) ribonucleoproteins delivered by lipid nanoparticles (rLNPs) reached sites of latent HIV-1 DNA. In these experiments humanized mice were created that permits long-term viral infection during treatment with antiretroviral therapy (ART). ART induces HIV-1 latency. Mice were irradiated then engrafted with human hematopoietic stem cells (HSC) in NOD.Cg-Prkdc^{scid I}12rgt^{mlWjl}/SzJ (NSG) mice. The CD34+HSC were isolated from cord blood by a single intrahepatic injection.

The presence of human immunocytes in blood was the mice was confirmed by flow cytometry. Four months after humanization animals were infected with HIV-1_{NL4-3} at 10⁴ tissue culture infection dose₅₀(TCID₅₀)/animal for 14 days. Mice were left HIV-1 infected and untreated (group 1): HIV-1 infected and ART (combinations of dolutegravir, tenofovir, and emtricitabine in food pellets, group 2): CRISPR-Cas9 (TatDE rLNPs, group 3) or both (group 4). The study scheme shows the times of infection and treatments. After viral infection, animals were followed for ten weeks and treated as outlined in the experimental scheme then sacrificed.

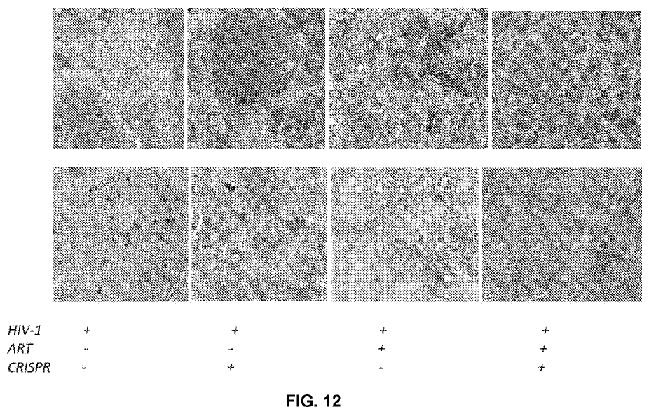

Human cell reconstitution was affirmed in spleen of mice by immunohistochemical staining with human HLA-DR antibodies (FIG. 12). Anatomical localizations and lymphocyte prominence were confirmed by human cell penetration into the white and red pulp and germinal centers of spleen. These were enriched with human cells with anatomical distinctions in the germinal centers. Productive HIV-1 infection was confirmed by HIV-1p24 staining as shown by large numbers of stained cells. Infection was highest in lymphoid compartments. HIV-1p24 staining was attenuated by ART with or without rLNP delivered CRISPR-Cas9 TatDE.

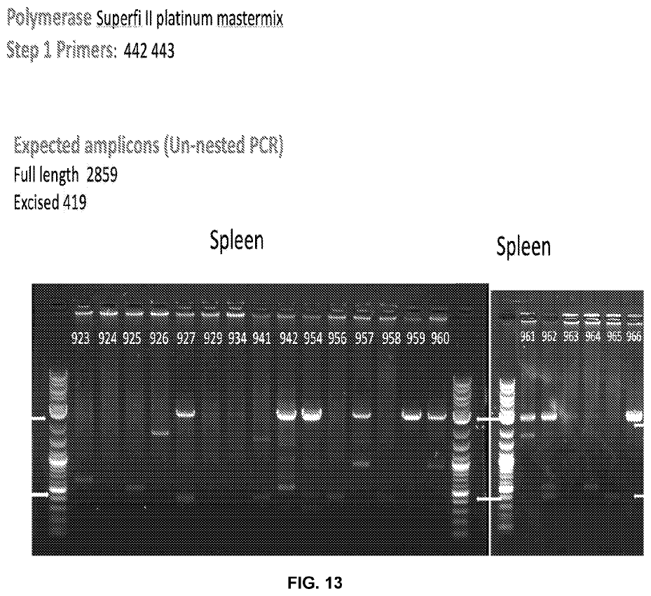

For proviral DNA elimination the CRISPR-Cas9 TatDE gene editing platform was deployed for gRNAs that cause cleavage of the viral genome at the highly conserved HIV-1 regions. This strategy allowed for the removal of the large intervening DNA fragments across the viral genome and mitigated any chance for the emergence of virus escape mutants. Results from cell culture and animal adoptive infection studies showed the absence of replication competent HIV-1 in the spleen of animals with no rebound that could be attributed to virus escape. The choice for the use of rLNP was based on efficient excision of HIV-1 DNA using CRISPR-Cas9 in infected cell lines. The results in this study verified the bioavailability of the disclosed gene editing molecules of the NSG humanized mice. See also, FIG. 13 showing excision of HIV-1 DNA by CRISPR-Cas9 in HIV-1 infected humanized mice.

What is claimed is:

1. A lipid nanoparticle, comprising a plurality of lipids and a CRISPR nucleic acid complementary to a HIV-1 gene.

2. The lipid nanoparticle of claim 1, wherein the lipid nanoparticle comprises cationic lipids, zwitterionic lipids, cholesterol, and PEG-lipid conjugates.

3. The lipid nanoparticle of claim 1, wherein the lipid nanoparticle comprises DMG-PEG2500, ionizable lipids, DSPC, cholesterol, and a stabilizer.

4. The lipid nanoparticle of claim 1, wherein the lipid nanoparticle comprises DSPE-PEG₂₀₀₀ and/or DMP-PEG₂₀₀₀, DOPE, cholesterol, DOTAP.

5. The lipid nanoparticle of claim 4, wherein the lipid nanoparticle comprises DSPE-PEG2000, DOPE, Cholesterol, DMG-PEG, and DOTAP, and wherein the molar percentages are about 5% to about 15%, about 5% to about 15%, about 20 to about 30%, about 1% to about 5%, and about 40 to about 60%, respectively.

6. The lipid nanoparticle of claim 1, comprising a crRNA sequence that is complementary to a plurality of nucleic acids of a consensus sequence of an HIV-1 gene selected from the group consisting of: tat, rev, env-gp41, gag-p1, gag-p6, vif, vpr, vpu, and nef.

7. The lipid nanoparticle of claim 1, wherein the nucleic acid sequence comprises two crRNA sequences, each sequence complementary to a plurality of nucleic acids of a consensus sequence of an HIV-1 gene selected from the group consisting of: tat, rev, env-gp41, gag-p1, gag-p6, vif, vpr, vpu, and nef; wherein the crRNA sequences are not complementary to the same sequences.

8. The lipid nanoparticle of claim 6, wherein the crRNA sequence is adjacent to a PAM sequence.

9. The lipid nanoparticle of claim 6, wherein the crRNA sequence is complementary to a plurality of nucleic acids of an overlapping sequence.

10. The lipid nanoparticle of claim 9, wherein the overlapping sequence is part of a nucleic acid sequence of at least two HIV-1 genes selected from the group consisting of: tat, rev, env-gp41, gag-p1, gag-p6, vif, vpr, vpu, and nef.

11. The lipid nanoparticle of claim 9, wherein the overlapping sequence is part of a nucleic acid sequence of at least three HIV-1 genes selected from the group consisting of: tat, rev, env-gp41, gag-p1, gag-p6, vif, vpr, vpu, and nef.

12. The lipid nanoparticle of claim 9, wherein the overlapping exon is part of a nucleic acid sequence selected from the group consisting of tat (exon 1, nucleic acids 5831-6045; exon 2, nucleic acids 8379-8469), rev (exon 1, nucleic acids 5970-6045; or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417).

13. The lipid nanoparticle of claim 9, wherein the overlapping sequence is nucleic acids 7758-8795 of HIV-1 gene gp41-env, exon 2 (nucleic acids 8379-8469) of HIV-1 gene tat, and exon 2 (nucleic acids 8379-8653) of HIV-1 gene rev.

14. The lipid nanoparticle of claim 9, wherein the overlapping exon is exon 1 (nucleic acids 5831-6045) of HIV-1 gene tat, and exon 1 (nucleic acids 5970-6045) of HIV-1 gene rev.

15. The lipid nanoparticle of claim 6, wherein the crRNA has a sequence at least 80%, 85%, 90%, or 95% identical to SEQ ID NO: 1.

16. The lipid nanoparticle of claim 6, wherein the crRNA has a sequence at least 80%, 85%, 90%, or 95% identical to SEQ ID NO: 2.

17. The lipid nanoparticle of claim 6, wherein the crRNA has a sequence at least 80%, 85%, 90%, or 95% identical to SEQ ID NO: 3.

18. The lipid nanoparticle of claim 6, wherein the crRNA has a sequence at least 80%, 85%, 90%, or 95% identical to SEQ ID NO: 4.

19. The lipid nanoparticle of claim 6, wherein the crRNA has a sequence at least 80%, 85%, 90%, or 95% identical to SEQ ID NO: 5.

20. The lipid nanoparticle of claim 6, wherein the crRNA has a sequence at least 80%, 85%, 90%, or 95% identical to SEQ ID NO: 6.

21. The lipid nanoparticle of claim 6, wherein the crRNA has a sequence at least 80%, 85%, 90%, or 95% identical to SEQ ID NO: 7.

22. The lipid nanoparticle of claim 6, wherein the crRNA has a sequence at least 80%, 85%, 90%, or 95% identical to SEQ ID NO: 8.

23. The lipid nanoparticle of claim 6, wherein the crRNA has a sequence according to SEQ ID NO: 1.

24. The lipid nanoparticle of claim 6, wherein the crRNA has a sequence according to SEQ ID NO: 2.

25. The lipid nanoparticle of claim 6, wherein the crRNA has a sequence according to SEQ ID NO: 3.

26. The lipid nanoparticle of claim 6, wherein the crRNA has a sequence according to SEQ ID NO: 4.

27. The lipid nanoparticle of claim 6, wherein the crRNA has a sequence according to SEQ ID NO: 5.

28. The lipid nanoparticle of claim 6, wherein the crRNA has a sequence according to SEQ ID NO: 6.

29. The lipid nanoparticle of claim 6, wherein the crRNA has a sequence according to SEQ ID NO: 7.

30. The lipid nanoparticle of claim 6, wherein the crRNA has a sequence according to SEQ ID NO: 8.

31. The lipid nanoparticle of claim 6, wherein the nucleic acid encodes for a TatDE crRNA.

32. The lipid nanoparticle of claim 31, wherein the TatDE crRNAs comprise SEQ ID NO: 2 and SEQ ID NO: 3.

33. The lipid nanoparticle of claim 1, wherein the nucleic acid sequence further comprises a tracrRNA sequence.

34. The lipid nanoparticle of claim 1, wherein the nucleic acid sequence further comprises a sequence that encodes a Cas protein.

35. The lipid nanoparticle of claim 33, wherein the Cas protein is a Cas9, CasPhi (Cas), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas12a (Cpf1), Cas12b, Cas12e, Cas13a, Cas13, Cas13c, or Cas13d.

36. The lipid nanoparticle of claim 35, wherein the Cas protein is a Cas9 protein.

37. The lipid nanoparticle of claim 36, where the nucleic acid encoding for Cas9 is a vector and the nucleic acid encoding for TatDE crRNAs is a vector.

38. The lipid nanoparticle of claim 36, where the nucleic acid encoding for Cas9 is a mRNA and the nucleic acid encoding for TatDE crRNAs is a mRNA.

39. The lipid nanoparticle of claim 1, wherein the nucleic acid sequence is a DNA sequence.

40. The lipid nanoparticle of claim 1, wherein the nucleic acid sequence is a RNA sequence.

41. A pharmaceutical composition, comprising (a) the lipid nanoparticle according to any one of claims 1-40, and (b) a pharmaceutically acceptable excipient.

42. The pharmaceutical composition of claim 41, comprising (a) a nucleic acid comprising TatD and (b) a nucleic acid comprising TatH (TatD/H).

43. The pharmaceutical composition of claim 41, comprising (a) a nucleic acid comprising TatD and (b) a nucleic acid comprising TatE (TatD/E).

44. The pharmaceutical composition of claim 41, comprising (a) a nucleic acid comprising TatE and (b) a nucleic acid comprising TatH (TatE/H).

45. The pharmaceutical composition of claim 41, comprising (a) a nucleic acid comprising TatD and (b) a nucleic acid comprising TatA2 (TatA2/D).

46. The pharmaceutical composition of claim 41, comprising a nucleic acid comprising TatD/tracrRNA and (b) a nucleic acid comprising TatH/tracrRNA.

47. The pharmaceutical composition of claim 41, comprising (a) a nucleic acid comprising TatD/tracrRNA and (b) a nucleic acid comprising TatE/tracrRNA.

48. The pharmaceutical composition of claim 41, comprising (a) a nucleic acid comprising TatE/tracrRNA and (b) a nucleic acid comprising TatH/tracrRNA.

49. The pharmaceutical composition of claim 41, comprising (a) a nucleic acid comprising TatD/tracrRNA and (b) a nucleic acid comprising TatA2/tracrRNA.

50. A pharmaceutical composition, comprising (a) the lipid nanoparticle according to any one of claim 1, and (b) a pharmaceutically acceptable excipient.

51. A method of disrupting the transcription of an exon of an HIV-1 sequence in an individual in need thereof, comprising administering to the individual the lipid nanoparticle according to any one of claims 1-40.

52. A method of excising all or a portion of an HIV-1 sequence in an individual in need thereof, comprising administering to the individual the lipid nanoparticle according to any one of claims 1-40.

53. A method of treating an HIV-1 infection in an individual in need thereof, comprising administering to the individual the lipid nanoparticle according to any one of claims 1-40.

54. A method of preventing, treating, and/or eradicating a disease in a subject in need thereof, said method comprising administering to said subject a lipid nanoparticle of any of claims 1-40.

55. A method of preventing an HIV-1 infection in an individual in need thereof, comprising prophylactically administering to the individual the lipid nanoparticle according to any one of claims 1-40.

56. A method of preventing transmission of an HIV-1 virus from a first individual to a second individual, comprising administering to the first individual the lipid nanoparticle according to any one of claims 1-40.

57. The method of claim 55, wherein the first individual is a pregnant woman and the second individual is a child.

58. A composition comprising a lipid nanoparticle according to any one of claims 1-40 for disrupting the transcription of an exon of an HIV-1 sequence in an individual in need thereof, comprising administering to the individual the composition.

59. A composition comprising a lipid nanoparticle according to any one of claims 1-40 for excising all or a portion of an HIV-1 sequence in an individual in need thereof, comprising administering to the individual the composition.

60. A composition comprising a lipid nanoparticle according to any one of claims 1-40 for treating an HIV-1 infection in an individual in need thereof, comprising administering to the individual the composition.

61. A composition comprising a lipid nanoparticle according to any one of claims 1-40 for preventing, treating, and/or eradicating a disease in a subject in need thereof, comprising administering to said subject the composition.

62. A composition comprising a lipid nanoparticle according to any one of claims 1-40 for preventing an HIV-1 infection in an individual in need thereof, comprising prophylactically administering to the individual the composition.

63. A composition comprising a lipid nanoparticle according to any one of claims 1-40 for preventing transmission of an HIV-1 virus from a first individual to a second individual, comprising administering to the first individual the composition.

64. A lipid nanoparticle of claim 1, further comprising a diagnostic agent.

65. The nanoparticle of claim 64, wherein the diagnostic agent is a MRI contrast agent, a fluorescent dye, or a nuclear medicine agent.

66. The nanoparticle of claim 64, wherein the diagnostic agent is a radiolabeled europium doped cobalt ferrite nanoparticle (177Lu/89ZrCFEu nanoparticle).

67. A method of diagnosing an individual in need of HIV therapy, comprising administering to the individual the lipid nanoparticle according to any one of claims 64-66.

68. A composition comprising a lipid nanoparticle according to any one of claims 64-66 for diagnosing an individual in need of HIV therapy, comprising administering to the individual the composition.


SEQUENCE LISTING (30 sequences)

SEQ ID NO: 1 - RNA, 20 nt, Artificial Sequence
  Description of Artificial Sequence Synthetic oligonucleotide
uagauccuaaccuagagccc

SEQ ID NO: 2 - RNA, 20 nt, Artificial Sequence
  Description of Artificial Sequence Synthetic oligonucleotide
ucuccuauggcaggaagaag

SEQ ID NO: 3 - RNA, 20 nt, Artificial Sequence
  Description of Artificial Sequence Synthetic oligonucleotide
gaaggaaucgaagaagaagg

SEQ ID NO: 4 - RNA, 20 nt, Artificial Sequence
  Description of Artificial Sequence Synthetic oligonucleotide
gaaagaaucgaagaaggagg

SEQ ID NO: 5 - RNA, 20 nt, Artificial Sequence
  Description of Artificial Sequence Synthetic oligonucleotide
ccgauuccuucgggccuguc

SEQ ID NO: 6 - RNA, 20 nt, Artificial Sequence
  Description of Artificial Sequence Synthetic oligonucleotide
ucuccgcuucuuccugccau

SEQ ID NO: 7 - RNA, 20 nt, Artificial Sequence
  Description of Artificial Sequence Synthetic oligonucleotide
gcuuaggcaucuccuauggc

SEQ ID NO: 8 - RNA, 20 nt, Artificial Sequence
  Description of Artificial Sequence Synthetic oligonucleotide
ggcucuagguuaggaucuac

SEQ ID NO: 9 - DNA, 20 bp, Human immunodeficiency virus
tagatcctaacctagagccc

SEQ ID NO: 10 - DNA, 20 bp, Human immunodeficiency virus
tctcctatggcaggaagaag

SEQ ID NO: 11 - DNA, 20 bp, Human immunodeficiency virus
gaaggaatcgaagaagaagg

SEQ ID NO: 12 - DNA, 20 bp, Human immunodeficiency virus
gaaagaatcgaagaaggagg

SEQ ID NO: 13 - DNA, 20 bp, Human immunodeficiency virus
ccgattccttcgggcctgtc

SEQ ID NO: 14 - DNA, 20 bp, Human immunodeficiency virus
tctccgcttcttcctgccat

SEQ ID NO: 15 - DNA, 20 bp, Human immunodeficiency virus
gcttaggcatctcctatggc

SEQ ID NO: 16 - DNA, 20 bp, Human immunodeficiency virus
ggctctaggttaggatctac

SEQ ID NO: 17 - DNA, 75 bp, Human immunodeficiency virus
cttaggcatctcctatggcaggaagaagcggagacagcgacgaagacctcctcaagacagtcagactaatcaagt

SEQ ID NO: 18 - DNA, 75 bp, Artificial Sequence
  Description of Artificial Sequence Synthetic oligonucleotide
  modified_base (26)..(29): a, c, g, t, unknown or other
cttaggcatctcctatggcaggaagnnnnaagcggagacagcgacgaagacctcctcaagacagtcagactaatc

SEQ ID NO: 19 - DNA, 75 bp, Artificial Sequence
  Description of Artificial Sequence Synthetic oligonucleotide
  modified_base (26)..(27): a, c, g, t, unknown or other
cttaggcatctcctatggcaggaagnnaagcggagacagcgacgaagacctcctcaagacagtcagactaatcaa

SEQ ID NO: 20 - DNA, 75 bp, Artificial Sequence
  Description of Artificial Sequence Synthetic oligonucleotide
  modified_base (26)..(33): a, c, g, t, unknown or other
cttaggcatctcctatggcaggaagnnnnnnnnaagcggagacagcgacgaagacctcctcaagacagtcagact

SEQ ID NO: 21 - DNA, 63 bp, Artificial Sequence
  Description of Artificial Sequence Synthetic oligonucleotide
cttaggcatctcctatggcaggaagcgacgaagacctcctcaagacagtcagactaatcaagt

SEQ ID NO: 22 - DNA, 75 bp, Human immunodeficiency virus
cttcccagccccgaggggacccgacaggcccgaaggaatcgaagaagaaggtggagagcgagacagagacagatc

SEQ ID NO: 23 - DNA, 63 bp, Artificial Sequence
  Description of Artificial Sequence Synthetic oligonucleotide
cttcccagccccgaggggacccgacatcgaagaagaaggtggagagcgagacagagacagatc

SEQ ID NO: 24 - DNA, 75 bp, Artificial Sequence
  Description of Artificial Sequence Synthetic oligonucleotide
  modified_base (26)..(29): a, c, g, t, unknown or other
cttcccagccccgaggggacccgacnnnnaggcccgaaggaatcgaagaagaaggtggagagcgagacagagaca

SEQ ID NO: 25 - DNA, 71 bp, Artificial Sequence
  Description of Artificial Sequence Synthetic oligonucleotide
cttcccagccccgaggggaccaggcccgaaggaatcgaagaagaaggtggagagcgagacagagacagatc

SEQ ID NO: 26 - DNA, 75 bp, Artificial Sequence
  Description of Artificial Sequence Synthetic oligonucleotide
  modified_base (26)..(35): a, c, g, t, unknown or other
cttcccagccccgaggggacccgacnnnnnnnnnnaggcccgaaggaatcgaagaagaaggtggagagcgagaca

SEQ ID NO: 27 - protein, 19 aa, Human immunodeficiency virus
MTKALGISYGRKKRRQRRR

SEQ ID NO: 28 - protein, 20 aa, Artificial Sequence
  Description of Artificial Sequence Synthetic polypeptide
MTKALGISYGRKKGRRQRRR

SEQ ID NO: 29 - protein, 13 aa, Human immunodeficiency virus
RPEGIEEEGGERD

SEQ ID NO: 30 - protein, 9 aa, Artificial Sequence
  Description of Artificial Sequence Synthetic polypeptide
RPEGIEERD